(19) United States
(12) Patent Application Publication
(10) Pub. No.: US 2008/0206841 A1
(43) Pub. Date: Aug. 28, 2008

(54) Nucleic acid sequences from Chlorella vulgaris and uses thereof

(76) Inventors: Raghunath V. Lalgudi, Clayton, MO (US); Philip W. Miller, Ballwin, MO (US); Keith O'Connell, Kirkwood, MO (US)

Correspondence Address: ARNOLD & PORTER, LLP, 555 TWELFTH STREET, N.W., ATTN: IP DOCKETING, WASHINGTON, DC, 20004, US

(21) Appl. No.: 11/239,592

(22) Filed: Sep. 30, 2005

Related U.S. Application Data
(63) Continuation of application No. 09/540,215, filed on Apr. 3, 2000, now abandoned.
(60) Provisional application No. 60/128,436, filed on Apr. 6, 1999.

(51) Int. Cl. C12N 1/12; C07H 21/00

(52) U.S. Cl. 435/257.2; 536/23.7

(57) ABSTRACT

Expressed Sequence Tags (ESTs) isolated from the unicellular green algae, Chlorella vulgaris, are disclosed. The invention encompasses nucleic acid molecules that encode Chlorella protein homologs and fragments thereof. In addition, antibodies capable of binding the proteins are encompassed by the present invention. The disclosed ESTs have particular utility in isolating genes and promoters, identifying and mapping the genes involved in developmental and metabolic pathways, and determining gene function. The ESTs provide a unique molecular tool for the targeting and isolation of novel genes for plant protection and improvement. The invention also relates to methods of using the disclosed nucleic acid molecules, proteins, fragments of proteins, and antibodies, for example, for gene identification and analysis, and preparation of constructs.

### CROSS-REFERENCE TO RELATED APPLICATIONS

This application claims priority under 35 U.S.C §119(e) of U.S. Provisional Application Ser. No. 60/128,436 filed on Apr. 6, 1999, the entire content of which is incorporated herein by reference.

### FIELD OF THE INVENTION

The present invention is in the field of molecular biology; more particularly, the present invention relates to nucleic acid sequences from the unicellular green algae, Chlorella vulgaris. The invention encompasses nucleic acid molecules that encode proteins and fragments of proteins. In addition, proteins and fragments of proteins so encoded and antibodies capable of binding the proteins are encompassed by the present invention. The invention also relates to methods of using the disclosed nucleic acid molecules, proteins, fragments of proteins, and antibodies, for example, for gene identification and analysis, and preparation of constructs.

### BACKGROUND OF THE INVENTION

I. Chlorella vulgaris

The present invention relates in part to DNA sequences from cDNA libraries from the unicellular green algae, Chlorella vulgaris. The green algal genus Chlorella includes a variety of species (Fott and Novakova, In: Studies in Phycology: A Monograph of the Genus Chlorella, Fott, B. (ed.), Prag: Verlag Acad. Sissensch, pp. 10-74 (1969), herein incorporated by reference in its entirety), some of which have long been served as model organisms in plant physiological and biochemical studies (Govindjee and Braun, In: Algal Physiology and Biochemistry, W. D. P. Stewart (ed.), University of California Press, Berkeley and Los Angeles, pp. 346-390, herein incorporated by reference in its entirety). Chlorella belongs to the eucaryotic cell category of algae and lives in fresh water as a single celled plant Its size is approximately 2-8 microns in diameter. The name Chlorella derives from two Latin words meaning ‘leaf’ (green) and ‘small’, referring to the unusually high content of chlorophyll which gives Chlorella its characteristic deep emerald-green color. Chlorella is also rich in protein, vitamins, minerals, “C.G.F.” (Chlorella Growth Factor) and other beneficial substances. Unicellular green algae Chlorella are currently being used to produce compounds of commercial value (Behrens et al., J. Applied Phycology 6: 113-122 (1994); Running et al., J. Applied Phycology 6: 99-104 (1994), both of which are herein incorporated by reference in their entirety).

It is generally believed that land plants evolved from green algae (Graham, J. Plant Res. 109: 241-251 (1996), herein incorporated by reference in its entirety) and that during this revolution, extensive rearrangements occurred within the chloroplast genome. The complete nucleotide sequence of the chloroplast genome (150613 bp) from the unicellular green alga Chlorella vulgaris has been determined (Wakasugi, et al., Proc. Natl. Acad. Sci. USA 94:5967-5972 (1997), herein incorporated by reference in its entirety). The chloroplast genome of Chlorella vulgaris contains one copy of rRNA gene consisting of 16S, 23S, and 5S rRNA genes; thirty one tRNA gene, sixty-nine protein genes; eight ORFs conserved with those found in land chloroplasts; two adjacent genes homologous to bacterial genes (minD and minE) involved in cell division; genes encoding ribosomal proteins L5, L12, L19 and S9; and two long ORF's related to ycf1 and ycf2 that are exclusively found in land plants (Wakasugi, et al., Proc. Natl. Acad. Sci. USA 94: 5967-5972 (1997), herein incorporated by reference in its entirety). Chlorella vulgaris is closer to land plants than the red and brown algae.

The genome of the unicellular green alga Chlorella vulgaris is only 38.8 Mb and consists of 16 chromosomes ranging from 980 kb to 4.0 Mb in size. (Higashiyama and Yamada, Nucleic Acid Res. 19: 6191-6195 (1991), herein incorporated by reference in its entirety). Each chromosome can be resolved by pulse-field gel electrophoresis. The smallest chromosome of this organism (Chromosome I, 980 kb in size) can be routinely isolated intact in large quantities. Restriction mapping and sequences analyses revealed that the telomeres of this chromosme consist of 5′-TTTAGGG repeats running from the centomere towards the termini; this sequence is identical to those reported for several higher plants including maize, Arabidopsis thaliana and tomato (Higashiyama, et al., Mol. Gen. Genet. 246: 29-36 (1995), herein incorporated by reference in its entirety). A set of overlapping cosmid clones (contig) have been constructed for Chlorella chromosome I (Noutoshi, et al., Nucleic Acid Res. 26:3900-3907 (1998), herein incorporated by reference in its entirety).

### II. Expressed Sequence Tag Nucleic Acid Molecules

Expressed sequence tags, or ESTs, are short sequences of randomly selected clones from a cDNA (or complementary DNA) library which are representative of the cDNA inserts of these randomly selected clones (McCombie, et al., Nature Genetics, 1:124-130 (1992); Kurata, et al., Nature Genetics, 8: 365-372 (1994); Okubo, et al., Nature Genetics, 2: 173-179 (1992), all of which are herein incorporated by reference in their entirety). The randomly selected clones comprise inserts that can represent a copy of up to the full length of a mRNA transcript.

Using conventional methodologies, cDNA libraries can be constructed from the mRNA (messenger RNA) of a given tissue or organism using poly dT primers and reverse transcriptase (Efstratiadis, et al., Cell 7:279-288 (1976); Higuchi, et al., Proc. Natl. Acad. Sci. (U.S.A.) 73:3146-3150 (1976); Maniatis, et al., Cell 8:163 (1976); Land, et al., Nucleic Acids Res. 9:2251-2266 (1981); Okayama, et al., Mol. Cell. Biol. 2:161-170 (1982); Gubler, et al., Gene 25:263 (1983), all of which are herein incorporated by reference in their entirety).

Several methods may be employed to obtain full-length cDNA constructs. For example, terminal transferase can be used to add homopolymeric tails of dC residues to the free 3′ hydroxyl groups (Land, et al., Nucleic Acids Res. 9:2251-2266 (1981), herein incorporated by reference in its entirety). This tail can then be hybridized by a poly dG oligo which can act as a primer for the synthesis of full length second strand cDNA (Okayama and Berg, Mol. Cell. Biol. 2:161-170 (1982), herein incorporated by reference in its entirety), report a method for obtaining full length cDNA constructs. This method has been simplified by using synthetic primer-adapters that have both homopolymeric tails for priming the synthesis of the first and second strands and restriction sites for cloning into plasmids (Coleclough, et al., Gene 34:305-314 (1985), herein incorporated by reference in its entirety) and bacteriophage vectors (Krawinkel, et al., Nucleic Acids Res. 14:1913 (1986); Han, et al., Nucleic Acids Res. 15:6304 (1987), all of which are herein incorporated by reference in their entirety).

These strategies have been coupled with additional strategies for isolating rare mRNA populations. For example, a typical mammalian cell contains between 10,000 and 30,000 different mRNA sequences (Davidson, Gene Activity in Early Development, 2nd ed., Academic Press, New York (1976), herein incorporated by reference in its entirety). The number of clones required to achieve a given probability that a low-abundance mRNA will be present in a cDNA library is N=(1 n(1−P))/(1 n(1−1/n)) where N is the number of clones required, P is the probability desired, and 1/n is the fractional proportion of the total mRNA that is represented by a single rare mRNA. (Sambrook, et al., Molecular Cloning: A Laboratory Manual, 2nd ed., Cold Spring Harbor Laboratory Press (1989), herein incorporated by reference in its entirety.).

A method to enrich preparations of mRNA for sequences of interest is to fractionate by size. One such method is to fractionate by electrophoresis through an agarose gel (Pennica, et al., Nature 301:214-221 (1983), herein incorporated by reference in its entirety). Another such method employs sucrose gradient centrifugation in the presence of an agent, such as methylmercuric hydroxide, that denatures secondary structure in RNA (Schweinfest, et al., Proc. Natl. Acad. Sci. (U.S.A.) 79:4997-5000 (1982), herein incorporated by reference in its entirety).

A frequently adopted method is to construct equalized or normalized cDNA libraries (Ko, Nucleic Acids Res. 18:5705-5711 (1990); Patanjali, S. R et al., Proc. Natl. Acad. Sci. (U.S.) 88:1943-1947 (1991), all of which are herein incorporated by reference in their entirety). Typically, the cDNA population is normalized by subtractive hybridization (Schmid, et al., J. Neurochem 48:307-312 (1987); Fargnoli, et al., Anal. Biochem. 187:364-373 (1990); Travis, et al., Proc. Natl. Acad. Sci (U.S.A.) 85:1696-1700 (1988); Kato, Eur. J. Neurosci. 2:704 (1990); and Schweinfest, et al., Genet. Anal. Tech Appl. 7:64 (1990), all of which are herein incorporated by reference in their entirety). Subtraction represents another method for reducing the population of certain sequences in the cDNA library (Swaroop, et al., Nucleic Acids Res. 19:1954 (1991), herein incorporated by reference in its entirety).

ESTs can be sequenced by a number of methods. Two basic methods may be used for DNA sequencing, the chain termination method of Sanger et al., Proc. Natl. Acad. Sci. (U.S.A.) 74: 5463-5467 (1977), herein incorporated by reference in its entirety and the chemical degradation method of Maxam and Gilbert, Proc. Nat. Acad. Sci. (U.S.A.) 74: 560-564 (1977), herein incorporated by reference in its entirety. Automation and advances in technology such as the replacement of radioisotopes with fluorescence-based sequencing have reduced the effort required to sequence DNA (Craxton, Methods, 2: 20-26 (1991); Ju et al., Proc. Natl. Acad. Sci. (U.S.A.) 92: 4347-4351 (1995); Tabor and Richardson, Proc. Natl. Acad. Sci. (U.S.A.) 92: 6339-6343 (1995), all of which are herein incorporated by reference in their entirety). Automated sequencers are available from, for example, Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997), all of which are herein incorporated by reference in their entirety).

ESTs longer than 150 base pairs have been found to be useful for similarity searches and mapping. (Adams, et al., Science 252:1651-1656 (1991), herein incorporated by reference.) ESTs, which can represent copies of up to the full length transcript, may be partially or completely sequenced. Between 150-450 nucleotides of sequence information is usually generated as this is the length of sequence information that is routinely and reliably produced using single run sequence data. Typically, only single run sequence data is obtained from the cDNA library (Adams, et al., Science 252:1651-1656 (1991), herein incorporated by reference in its entirety). Automated single run sequencing typically results in an approximately 2-3% error or base ambiguity rate. (Boguski, et al., Nature Genetics, 4:332-333 (1993), herein incorporated by reference in its entirety).

EST databases have been constructed or partially constructed from, for example, C. elegans (McCombrie, et al., Nature Genetics 1:124-131 (1992), herein incorporated by reference in its entirety), human liver cell line HepG2 (Okubo, et al., Nature Genetics 2:173-179 (1992), herein incorporated by reference in its entirety), human brain RNA (Adams, et al., Science 252:1651-1656 (1991); Adams, et al., Nature 355:632-635 (1992), all of which are herein incorporated by reference in their entirety), Arabidopsis, (Newman, et al., Plant Physiol. 106:1241-1255 (1994), herein incorporated by reference in its entirety); and rice (Kurata, et al., Nature Genetics 8:365-372 (1994), herein incorporated by reference in its entirety).

### III. Sequence Comparisons

A characteristic feature of a DNA sequence is that it can be compared with other known DNA sequences. Sequence comparisons can be undertaken by determining the similarity of the test or query sequence with sequences in publicly available or propriety databases (“similarity analysis”) or by searching for certain motifs (“intrinsic sequence analysis”) (e.g. cis elements) (Coulson, Trends in Biotechnology 12: 76-80 (1994); Birren, et al, Genome Analysis, 1: 543-559 (1997), all of which are herein incorporated by reference in their entirety).

Similarity analysis includes database search and alignment. Examples of public databases include the DNA Database of Japan (DDBJ) (http://www.ddbj.nig.ac.jp/); Genebank (http://www.ncbi.nlm.nih.gov/web/Genbank/Index.htlm); and the European Molecular Biology Laboratory Nucleic Acid Sequence Database (EMBL) (http://www.ebi.ac.uk/ebi_docs/embl_db.html). A number of different search algorithms have been developed, one example of which are the suite of programs referred to as BLAST programs. There are five implementations of BLAST, three designed for nucleotide sequences queries (BLASTN, BLASTX, and TBLASTX) and two designed for protein sequence queries (BLASTP and TBLASTN) (Coulson, Trends in Biotechnology 12: 76-80 (1994); Birren et al., Genome Analysis 1: 543-559 (1997), all of which are herein incorporated by reference in their entirety).

BLASTN takes a nucleotide sequence (the query sequence) and its reverse complement and searches them against a nucleotide sequence database. BLASTN was designed for speed, not maximum sensitivity, and may not find distantly related coding sequences. BLASTX takes a nucleotide sequence, translates it in three forward reading frames and three reverse complement reading frames, and then compares the six translations against a protein sequence database. BLASTX is useful for sensitive analysis of preliminary (single-pass) sequence data and is tolerant of sequencing errors (Gish and States, Nature Genetics 3: 266-272 (1993), herein incorporated by reference in its entirety). BLASTN and BLASTX may be used in concert for analyzing EST data (Coulson, Trends in Biotechnology 12: 76-80 (1994); Birren et al., Genome Analysis 1: 543-559 (1997), all of which are herein incorporated by reference in their entirety).

Given a coding nucleotide sequence and the protein it encodes, it is often preferable to use the protein as the query sequence to search a database because of the greatly increased sensitivity to detect more subtle relationships. This is due to the larger alphabet of proteins (20 amino acids) compared with the alphabet of nucleic acid sequences (4 bases), where it is far easier to obtain a match by chance. In addition, with nucleotide alignments, only a match (positive score) or a mismatch (negative score) is obtained, but with proteins, the presence of conservative amino acid substitutions can be taken into account Here, a mismatch may yield a positive score if the non-identical residue has physical/chemical properties similar to the one it replaced. Various scoring matrices are used to supply the substitution scores of all possible amino acid pairs. A general purpose scoring system is the BLOSUM62 matrix (Henikoff and Henikoff, Proteins 17: 49-61 (1993), herein incorporated by reference in its entirety), which is currently the default choice for BLAST programs. BLOSUM62 is tailored for alignments of moderately diverged sequences and thus may not yield the best results under all conditions (Altschul, J. Mol. Biol. 36: 290-300 (1993), herein incorporated by reference in its entirety), uses a combination of three matrices to cover all contingencies. This may improve sensitivity, but at the expense of slower searches. In practice, a single BLOSUM62 matrix is often used but others (PAM40 and PAM250) may be attempted when additional analysis is necessary. Low PAM matrices are directed at detecting very strong but localized sequence similarities, whereas high PAM matrices are directed at detecting long but weak alignments between very distantly related sequences.

Homologues in other organisms are available that can be used for comparative sequence analysis. Multiple alignments are performed to study similarities and differences in a group of related sequences. CLUSTAL W is a multiple sequence alignment package available that performs progressive multiple sequence alignments based on the method of Feng and Doolittle, J. Mol. Evol. 25: 351-360 (1987), herein incorporated by reference in its entirety. Each pair of sequences is aligned and the distance between each pair is calculated; from this distance matrix, a guide tree is calculated, and all of the sequences are progressively aligned based on this tree. A feature of the program is its sensitivity to the effect of gaps on the alignment; gap penalties are varied to encourage the insertion of gaps in probable loop regions instead of in the middle of structured regions. Users can specify gap penalties, choose between a number of scoring matrices, or supply their own scoring matrix for both the pairwise alignments and the multiple alignments. CLUSTAL W for UNIX and VMS systems is available at: ftp.ebi.ac.uk. Another program is MACAW (Schuler et al., Proteins, Struct. Func. Genet. 9: 180-190 (1991), herein incorporated by reference in its entirety), for which both Macintosh and Microsoft Windows versions are available. MACAW uses a graphical interface, provides a choice of several alignment algorithms, and is available by anonymous ftp at: ncbi.nlm.nih.gov (directory/pub/macaw).

Sequence motifs are derived from multiple alignments and can be used to examine individual sequences or an entire database for subtle patterns. With motifs, it is sometimes possible to detect distant relationships that may not be demonstrable based on comparisons of primary sequences alone. Currently, the largest collection of sequence motifs in the world is. PROSITE (Bairoch and Bucher, Nucleic Acid Research 22: 3583-3589 (1994), herein incorporated by reference in its entirety). PROSITE may be accessed via either the ExPASy server on the World Wide Web or anonymous ftp site. Many commercial sequence analysis packages also provide search programs that use PROSITE data.

A resource for searching protein motifs is the BLOCKS E-mail server developed by S. Henikoff (Henikoff, Trends Biochem Sci. 18: 267-268 (1993); Henikoff and Henikoff, Nucleic Acid Research 19: 65656572 (1991); Henikoff and Henikoff, Proteins 17: 49-61 (1993), all of which are herein incorporated by reference in their entirety). BLOCKS searches a protein or nucleotide sequence against a database of protein motifs or “blocks.” Blocks are defined as short, ungapped multiple alignments that represent highly conserved protein patterns. The blocks themselves are derived from entries in PROSITE as well as other sources. Either a protein or nucleotide query can be submitted to the BLOCKS server; if a nucleotide sequence is submitted, the sequence is translated in all six reading frames and motifs are sought in these conceptual translations. Once the search is completed, the server will return a ranked list of significant matches, along with an alignment of the query sequence to the matched BLOCKS entries.

Conserved protein domains can be represented by two-dimensional matrices, which measure either the frequency or probability of the occurrences of each amino acid residue and deletions or insertions in each position of the domain. This type of model, when used to search against protein databases, is sensitive and usually yields more accurate results than simple motif searches. Two popular implementations of this approach are profile searches (such as GCG program ProfileSearch) and Hidden Markov Models (HMMs) (Krough et al., J. Mol. Biol. 235: 1501-1531 (1994); Eddy, Current Opinion in Structural Biology 6: 361-365 (1996), both of which are herein incorporated by reference in their entirety). In both cases, a large number of common protein domains have been converted into profiles, as present in the PROSITE library, or HHM models, as in the Pfam protein domain library (Sonnhammer et al., Proteins 28: 405-420 (1997), herein incorporated by reference in its entirety). Pfam contains more than 500 HMM models for enzymes, transcription factors, signal transduction molecules, and structural proteins. Protein databases can be queried with these profiles or HMM models, which will identify proteins containing the domain of interest. For example, HMMSW or HMMFS, two programs in a public domain package called HMMER (Sonnhammer et al., Proteins 28: 405-420 (1997), herein incorporated by reference in its entirety) can be used.

PROSITE and BLOCKS represent collected families of protein motifs. Thus, searching these databases entails submitting a single sequence to determine whether or not that sequence is similar to the members of an established family. Programs working in the opposite direction compare a collection of sequences with individual entries in the protein databases. An example of such a program is the Motif Search Tool, or MoST (Tatusov et al, Proc. Natl. Acad. Sci. 91:12091-12095 (1994), herein incorporated by reference in its entirety.) On the basis of an aligned set of input sequences, a weight matrix is calculated by using one of four methods (selected by the user); a weight matrix is simply a representation, position by position in an alignment, of how likely a particular amino acid will appear. The calculated weight matrix is then used to search the databases. To increase sensitivity, newly found sequences are added to the original data set, the weight matrix is recalculated, and the search is performed again. This procedure continues until no new sequences are found.

### SUMMARY OF THE INVENTION

The present invention provides a substantially purified nucleic acid molecule having a nucleic acid sequence selected from the group consisting of SEQ ID NO: 1 through SEQ ID NO: 3519 or complements thereof.

The present invention also provides a substantially purified nucleic acid molecule, the nucleic acid molecule capable of specifically hybridizing to a second nucleic acid molecule having a nucleic acid sequence selected from the group consisting of SEQ ID NO: 1 through SEQ ID NO: 3519 or complements thereof.

The present invention further provides a substantially purified protein, peptide, or fragment thereof encoded by a nucleic acid sequence selected from the group consisting of SEQ ID NO: 1 through SEQ ID NO:3519 or complements thereof.

The present invention also provides a substantially purified nucleic acid molecule encoding an Chlorella vulgaris protein homologue or fragment thereof, wherein the nucleic acid molecules comprises a nucleic acid sequence selected from the group consisting of SEQ ID NO: 1 through SEQ ID NO: 3519.

The present invention also provides a transformed cell having a nucleic acid molecule which comprises: (A) an exogenous promoter region which functions in the cell to cause the production of a mRNA molecule; which is linked to (B) a structural nucleic acid molecule, wherein the structural nucleic acid molecule comprises a nucleic acid sequence selected from the group consisting of SEQ ID NO: 1 through SEQ ID NO:3519 or complements thereof; which is linked to (C) a 3′ non-translated sequence that functions in the cell to cause termination of transcription and addition of polyadenylated ribonucleotides to a 3′ end of the mRNA molecule.

The present invention also provides a plant cell, a mammalian cell, a bacterial cell, an insect cell, a fungal cell and an algal cell transformed with a nucleic acid molecule of the present invention.

The present invention also provides a computer readable medium having recorded thereon one or more of the nucleotide sequences depicted in SEQ ID NO:1 through SEQ ID NO: 3519 or complements thereof.

### DETAILED DESCRIPTION OF THE INVENTION

### Agents of the Invention

(a) Nucleic Acid Molecules

Agents of the present invention include nucleic acid molecules and more specifically EST nucleic acid molecules or nucleic acid fragment molecules thereof. Fragment EST nucleic acid molecules may encode significant portion(s) of; or indeed most of, the EST nucleic acid molecule. Alternatively, the fragments may comprise smaller oligonucleotides (having from about 15 to about 250 nucleotide residues, and more preferably, about 15 to about 30 nucleotide residues).

In a preferred embodiment the nucleic acid molecules of the present invention are derived from a unicellular green alga and in an even more preferred embodiment the nucleic acid molecules of the present invention are derived from unicellular green algae belonging to the genus Chlorella In a particularly preferred embodiment the nucleic acid molecules of the present invention are derived from Chlorella vulgaris.

The term “substantially purified”, as used herein, refers to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by antibody (or to compete with another molecule for such binding). Alternatively, such an attribute may be catalytic, and thus involve the capacity of the agent to mediate a chemical reaction or response.

The agents of the present invention may also be recombinant. As used herein, the term recombinant means any agent (e.g. DNA, peptide etc.), that is, or results, however indirect, from human manipulation of a nucleic acid molecule.

It is understood that the agents of the present invention may be labeled with reagents that facilitate detection of the agent (e.g. fluorescent labels (Prober, et al., Science 238:336-340 (1987); Albarella et al, EP 144914, chemical labels (Sheldon et al, U.S. Pat. No. 4,582,789; Albarella et al., U.S. Pat. No. 4,563,417, modified bases (Miyoshi et al., EP 119448, all of which are herein incorporated by reference in their entirety).

It is further understood, that the present invention provides bacterial, viral, microbial, and plant cells comprising the agents of the present invention.

EST nucleic acid molecules or fragment EST nucleic acid molecules are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook, et al., In: Molecular Cloning A Laboratory Manual, 2nd Edition, Cold Spring Harbor Press, Cold Spring Harbor, N.Y. (1989), and by Haymes, et al. In: Nucleic Acid Hybridization, A Practical Approach, IRL Press, Washington, D.C. (1985), herein incorporated by reference in its entirety. Departres from complete complementarity are therefore permissible, as long as such departures do not completely preclude the capacity of the molecules to form a double-stranded structure. Thus, in order for an EST nucleic acid molecule or fragment EST nucleic acid molecule to serve as a primer or probe it need only be sufficiently complementary in sequence to be able to form a stable double-stranded structure under the particular solvent and salt concentrations employed.

Appropriate stringency conditions which promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, a nucleic acid of the present invention will specifically hybridize to one or more of the nucleic acid molecules set forth in SEQ ID NO: 1 through SEQ ID NO: 3519 or complements thereof under moderately stringent conditions, for example at about 2.0×SSC and about 65° C.

In a particularly preferred embodiment, a nucleic acid of the present invention will include those nucleic acid molecules that specifically hybridize to one or more of the nucleic acid molecules set forth in SEQ ID NO: 1 through SEQ ID NO: 3519 or complements thereof under high stringency conditions.

In one aspect of the present invention, the nucleic acid molecules of the present invention have one or more of the nucleic acid sequences set forth in SEQ ID NO: 1 through to SEQ ID NO:3519 or complements thereof. In another aspect of the present invention, one or more of the nucleic acid molecules of the present invention share between 100% and 90% sequence identity with one or more of the nucleic acid sequences set forth in SEQ ID NO: 1 through to SEQ ID NO:3519 or complements thereof. In a further aspect of the present invention, one or more of the nucleic acid molecules of the present invention share between 100% and 95% sequence identity with one or more of the nucleic acid sequences set forth in SEQ ID NO: 1 through to SEQ ID NO:3519 or complements thereof. In a more preferred aspect of the present invention, one or more of the nucleic acid molecules of the present invention share between 100% and 98% sequence identity with one or more of the nucleic acid sequences set forth in SEQ ID NO: 1 through to SEQ ID NO:3519 or complements thereof. In an even more preferred aspect of the present invention, one or more of the nucleic acid molecules of the present invention share between 100% and 99% sequence identity with one or more of the sequences set forth in SEQ ID NO: 1 through to SEQ ID NO:3519 or complements thereof. In a further, even more preferred aspect of the present invention, one or more of the nucleic acid molecules of the present invention exhibit 100% sequence identity with one or more nucleic acid molecules present within the cDNA library LIB191, herein designated (Monsanto Company, St Louis, Mo., United States of America).

The degeneracy of the genetic code, which allows different nucleic acid sequences to code for the same protein or peptide, is known in the literature. (U.S. Pat. No. 4,757,006, herein incorporated by reference in its entirety). As used herein a nucleic acid molecule is degenerate of another nucleic acid molecule when the nucleic acid molecules encode for the same amino acid sequences but comprise different nucleotide sequences. An aspect of the present invention is that the nucleic acid molecules of the present invention include nucleic acid molecules that are degenerate of those set forth in SEQ ID NO: 1 through to SEQ ID NO:3519 or complements thereof.

(b) Protein and Peptide Molecules

A class of agents comprises one or more of the protein or peptide molecules encoded by SEQ ID NO: 1 through SEQ ID NO:3519 or one or more of the protein or fragment thereof or peptide molecules encoded by other nucleic acid agents of the present invention. Protein and peptide molecules can be identified using known protein or peptide molecules as a target sequence or target motif in the BLAST programs of the present invention. In a preferred embodiment the protein or fragment molecules of the present invention are derived from Chlorella vulgaris. As used herein, the term “protein molecule” or “peptide molecule” includes any molecule that comprises five or more amino acids. It is well known in the art that proteins may undergo modification, including post-translational modifications, such as, but not limited to, disulfide bond formation, glycosylation, phosphorylation, or oligomerization. Thus, as used herein, the term “protein molecule” or “peptide molecule” includes any protein molecule that is modified by any biological or non-biological process. The terms “amino acid” and “amino acids” refer to all naturally occurring L-amino acids. This definition is meant to include norleucine, ornithine, homocysteine, and homoserine.

One or more of the protein or fragment of peptide molecules may be produced via chemical synthesis, or more preferably, by expressing in a suitable bacterial or eukaryotic host. Suitable methods for expression are described by Sambrook, et al., (In: Molecular Cloning, A Laboratory Manual, 2nd Edition, Cold Spring Harbor Press, Cold Spring Harbor, N.Y. (1989), herein incorporated by reference in its entirety), or similar texts.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein. Such molecules may be derivatized to contain carbohydrate or other moieties (such as keyhole limpet hemocyanin, etc.). Fusion protein or peptide molecule of the present invention are preferably produced via recombinant means.

Another class of agents comprise protein or peptide molecules encoded by SEQ ID NO: 1 through SEQ ID NO:3519 or, fragments or fusions thereof in which non-essential, or not relevant, amino acid residues have been added, replaced, or deleted. An example of such a homologue is the homologue protein of a plant, including but not limited to soybean, alfalfa, Arabidopsis, barley, cotton, corn, oat, oilseed rape, rice, canola, maize, ornamentals, sugarcane, sugarbeet, tomato, potato, wheat, and turf grasses. Such a homologue can be obtained by any of a variety of methods. Most preferably, as indicated above, one or more of the disclosed sequences (e.g., SEQ ID. NO: 1 through SEQ ID NO:3519 or complements thereof) will be used to define a pair of primers that may be used to isolate the homologue-encoding nucleic acid molecules from any desired species. Such molecules can be expressed to yield homologues by recombinant means.

In a preferred embodiment of the present invention, a Chlorella vulgaris protein or fragment thereof of the present invention is a homologue of another algal protein. In another preferred embodiment of the present invention, a Chlorella vulgaris protein or fragment thereof of the present invention is a homologue of a fungal protein. In another preferred embodiment of the present invention, a Chlorella vulgaris protein or fragment thereof of the present invention is a homologue of mammalian protein. In another preferred embodiment of the present invention, a Chlorella vulgaris protein or fragment thereof of the present invention is a homologue of a bacterial protein.

In a preferred embodiment of the present invention, the nucleic molecule of the present invention encodes a Chlorella vulgaris protein or fragment thereof where a Chlorella vulgaris protein or fragment thereof exhibits a BLAST probability score of greater than E-12, preferably a BLAST probability score of between about 1E-30 and about 1E-12, even more preferably a BLAST probability score of greater than 1E-30 with its homologue.

In another preferred embodiment of the present invention, the nucleic acid molecule encoding a Chlorella vulgaris protein or fragment thereof exhibits a % identity with its homologue of between about 25% and about 40%/, more preferably of between about 40 and about 70%, even more preferably of between about 70% and about 90% and even more preferably between about 90% and 99%. In another preferred embodiment, of the present invention, a Chlorella vulgaris protein or fragment thereof exhibits a % identity with its homologue of 100%.

In a preferred embodiment of the present invention, the nucleic molecule of the present invention encodes a Chlorella vulgaris protein or fragment thereof where the Chlorella vulgaris protein exhibits a BLAST score of greater than 120, preferably a BLAST score of between about 1450 and about 120, even more preferably a BLAST score of greater than 1450 with its homologue.

The degeneracy of the genetic code, which allows different nucleic acid sequences to code for the same protein or peptide, is known in the literature. (U.S. Pat. No. 4,757,006, herein incorporated by reference in its entirety). As used herein a nucleic acid molecule is degenerate of another nucleic acid molecule when the nucleic acid molecules encode for the same amino acid sequences but comprise different nucleotide sequences.

In an aspect of the present invention, one or more of the nucleic acid molecules of the present invention differ in nucleic acid sequence from those encoding a Chlorella vulgaris protein or fragment thereof in SEQ ID NO: 1 through SEQ ID NO: 3519 due to the degeneracy in the genetic code in that they encode the same protein but differ in nucleic acid sequence.

In another further aspect of the present invention, nucleic acid molecules of the present invention can comprise sequences, which differ from those encoding a protein or fragment thereof in SEQ ID NO: 1 through SEQ ID NO: 3519 due to fact that the different nucleic acid sequence encodes a protein having one or more conservative amino acid changes. It is understood that codons capable of coding for such conservative amino acid substitutions are known in the art.

It is well known in the art that one or more amino acids in a native sequence can be substituted with another amino acid(s), the charge and polarity of which are similar to that of the native amino acid, i.e., a conservative amino acid substitution, resulting in a silent change. Conserved substitutes for an amino acid within the native polypeptide sequence can be selected from other members of the class to which the naturally occurring amino acid belongs. Amino acids can be divided into the following four groups: (1) acidic amino acids, (2) basic amino acids, (3)-neutral polar amino acids, and (4) neutral nonpolar amino acids. Representative amino acids within these various groups include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine.

Conservative amino acid changes within the native polypeptides sequence can be made by substituting one amino acid within one of these groups with another amino acid within the same group. Biologically functional equivalents of the proteins or fragments thereof of the present invention can have 10 or fewer conservative amino acid changes, more preferably seven or fewer conservative amino acid changes, and most preferably five or fewer conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigen-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, obtain a protein with like properties. It is thus contemplated by the inventors that various changes may be made in the peptide sequences of the proteins or fragments of the present invention, or corresponding DNA sequences that encode said peptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157, 105-132 (1982), herein incorporated by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, 1982); these are isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (4.5).

In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference in its entirety, states that the greatest local average hydrophilicity of a protein, as govern by the hydrophilicity of its adjacent amino acids, correlates with a biological property of the protein.

As detailed in U.S. Pat. No. 4,554,101, the following hydrophilicity values have been assigned to amino acid residues: arginine (+3.0), lysine (+3.0), aspartate (+3.0±1), glutamate (+3.0±1), serine (+0.3), asparagine (+0.2), glutamine (+0.2), glycine (0), threonine (−0.4), proline (−0.5±1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4).

In making such changes, the substitution of amino acids whose hydrophilicity values are within ±2 is preferred, those which are within ±1 are particularly preferred, and those within ±0.5 are even more particularly preferred.

In a further aspect of the present invention, one or more of the nucleic acid molecules of the present invention differ in nucleic acid sequence from those encoding a Chlorella vulgaris protein or fragment thereof set forth in SEQ ID NO: 1 through SEQ ID. NO: 3519 or fragment thereof due to the fact that one or more codons encoding an amino acid has been substituted for a codon that encodes a nonessential substitution of the amino acid originally encoded.

(c) Antibodies

One aspect of the present invention concerns antibodies, single-chain antigen binding molecules, or other proteins that specifically bind to one or more of the protein or peptide molecules of the present invention and their homologues, fusions or fragments. Such antibodies may be used to quantitatively or qualitatively detect the protein or peptide molecules of the present invention. As used herein, an antibody or peptide is said to “specifically bind” to a protein or peptide molecule of the present invention if such binding is not competitively inhibited by the presence of non-related molecules. In a preferred embodiment the antibodies of the present invention bind to proteins of the present invention. In a more preferred embodiment the antibodies of the present invention bind to proteins derived from Chlorella vulgaris.

Nucleic acid molecules that encode all or part of the protein of the present invention can be expressed, via recombinant means, to yield protein or peptides that can in turn be used to elicit antibodies that are capable of binding the expressed protein or peptide. Such antibodies may be used in immunoassays for that protein. Such protein-encoding molecules, or their fragments may be a “fusion” molecule (i.e., a part of a larger nucleic acid molecule) such that, upon expression, a fusion protein is produced. It is understood that any of the nucleic acid molecules of the present invention may be expressed, via recombinant means, to yield proteins or peptides encoded by these nucleic acid molecules.

The antibodies that specifically bind proteins and protein fragments of the present invention may be polyclonal or monoclonal, and may comprise intact immunoglobulins, or antigen binding portions of immunoglobulins (such as (F(ab′), F(ab′)₂) fragments, or single-chain immunoglobulins producible, for example, via recombinant means). It is understood that practitioners are familiar with the standard resource materials which describe specific conditions and procedures for the construction, manipulation and isolation of antibodies (see, for example, Harlow and Lane, In Antibodies: A Laboratory Manual, Cold Spring Harbor Press, Cold Spring Harbor, N.Y. (1988), herein incorporated by reference in its entirety).

Murine monoclonal antibodies are particularly preferred. BALB/c mice are preferred for this purpose, however, equivalent strains may also be used. The animals are preferably immunized with approximately 25 μg of purified protein (or fragment thereof) that has been emulsified a suitable adjuvant (such as TiterMax adjuvant (Vaxcel, Norcross, Ga.)). Immunization is preferably conducted at two intramuscular sites, one intraperitoneal site, and one subcutaneous site at the base of the tail. An additional i.v. injection of approximately 25 μg of antigen is preferably given in normal saline three weeks later. After approximately 11 days following the second injection, the mice may be bled and the blood screened for the presence of anti-protein or peptide antibodies. Preferably, a direct binding Enzyme-Linked Immunoassay (ELISA) is employed for this purpose.

More preferably, the mouse having the highest antibody titer is given a third i.v. injection of approximately 25 μg of the same protein or fragment. The splenic leukocytes from this animal may be recovered 3 days later, and are then permitted to fuse, most preferably, using polyethylene glycol, with cells of a suitable myeloma cell line (such as, for example, the P3X63Ag8.653 myeloma cell line). Hybridoma cells are selected by culturing the cells under “HAT” (hypoxanthine-amiopterin-thymine) selection for about one weeks The resulting clones may then be screened for their capacity to produce monoclonal antibodies (“mAbs), preferably by direct ELISA.

In one embodiment, anti-protein or peptide monoclonal antibodies are isolated using a fusion of a protein, protein fragment, or peptide of the present invention, or conjugate of a protein, protein fragment, or peptide of the present invention, as immunogens. Thus, for example, a group of mice can be immunized using a fusion protein emulsified in Freund's complete adjuvant (e.g. approximately 50 μg of antigen per immunization). At three week intervals, an identical amount of antigen is emulsified in Freund's incomplete adjuvant and used to immunize the animals. Ten days following the third immunization, serum samples are taken and evaluated for the presence of antibody. If antibody titers are too low, a fourth booster can be employed. Polysera capable of binding the protein or peptide can also be obtained using this method.

In a preferred procedure for obtaining monoclonal antibodies, the spleens of the above-described immunized mice are removed, disrupted, and immune splenocytes are isolated over a ficoll gradient. The isolated splenocytes are fused, using polyethylene glycol with BALB/c-derived HGPRT (hypoxanthine guanine phosphoribosyl transferase) deficient P3×63xAg8.653 plasmacytoma cells. The fused cells are plated into 96-well microtiter plates and screened for hybridoma fusion cells by their capacity to grow in culture medium supplemented with hypothanthine, aminopterin and thymidine for approximately 2-3 weeks.

Hybridoma cells that arise from such incubation are preferably screened for their capacity to produce an immunoglobulin that binds to a protein of interest. An indirect ELISA may be used for this purpose. In brief, the supernatants of hybridomas are incubated in microtiter wells that contain immobilized protein. After washing, the titer of bound immunoglobulin can be determined using, for example, a goat anti-mouse antibody conjugated to horseradish peroxidase. After additional washing, the amount of immobilized enzyme is determined (for example through the use of a chromogenic substrate). Such screening is performed as quickly as possible after the identification of the hybridoma in order to ensure that a desired clone is not overgrown by non-secreting neighbors. Desirably, the fusion plates are screened several times since the rates of hybridoma growth vary. In a preferred sub-embodiment, a different antigenic form of immunogen may be used to screen the hybridoma. Thus, for example, the splenocytes may be immunized with one immunogen, but the resulting hybridomas can be screened using a different immunogen. It is understood that any of the protein or peptide molecules of the present invention may be used to raise antibodies.

As discussed below, such antibody molecules or their fragments may be used for diagnostic purposes. Where the antibodies are intended for diagnostic purposes, it may be desirable to derivatized them, for example with a ligand group (such as biotin) or a detectable marker group (such as a fluorescent group, a radioisotope or an enzyme).

The ability to produce antibodies that bind the protein or peptide molecules of the present invention permits the identification of mimetic compounds of those molecules. A “mimetic compound” is a compound that is not that compound, or a fragment of that compound, but which nonetheless exhibits an ability to specifically bind to antibodies directed against that compound.

It is understood that any of the agents of the present invention can be substantially purified and/or be biologically active and/or recombinant.

(d) Algal Constructs and Algal Transformants

The present invention also relates to an algal recombinant vector comprising exogenous genetic material. The present invention also relates to an algal cell comprising an algal recombinant vector. The present invention also relates to methods for obtaining a recombinant algal host cell comprising introducing into an algal host cell exogenous genetic material.

Exogenous genetic material is any genetic material, whether naturally occurring or otherwise, from any source that is capable of being inserted into any organism. Exogenous genetic material may be transferred into an algal cell. In a preferred embodiment the exogenous genetic material includes a nucleic acid molecule having a sequence selected from the group consisting of SEQ ID NO: 1 through SEQ ID NO: 3519 or complements thereof.

The algal recombinant vector may be any vector which can be conveniently subjected to recombinant DNA procedures. The choice of a vector will typically depend on the compatibility of the vector with the algal host cell into which the vector is to be introduced. The vector may be a linear or a closed circular plasmid. The vector system may be a single vector or plasmid or two or more vectors or plasmids which together contain the total DNA to be introduced into the genome of the algal host.

The algal vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the algal cell, is integrated into the genome and replicated together with the chromosome(s) into which it has been integrated. For integration, the vector may rely on the nucleic acid sequence of the vector for stable integration of the vector into the genome by homologous or nonhomologous recombination. Alternatively, the vector may contain additional nucleic acid sequences for directing integration by homologous recombination into the genome of the algal host. The additional nucleic acid sequences enable the vector to be integrated into the host cell genome at a precise location(s) in the chromosome(s). To increase the likelihood of integration at a precise location, there should be preferably two nucleic acid sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the algal host cell, and, furthermore, may be non-encoding or encoding sequences.

The vectors of the present invention preferably contain one or more selectable markers which permit easy selection of transformed cells. A selectable marker is a gene, the product of which confers upon an algal cell resistance to a compound to which the algal would otherwise be sensitive. The compound can be selected from the group consisting of antibiotics, fungicides, herbicides, and heavy metals. The selectable marker may be selected from any known or subsequently identified selectable markers, including markers derived from algal, fungal, and bacterial sources. Preferred selectable markers can be selected from the group including, but not limited to, amdS (acetamidase), argB (ornithine carbamoyltransferase), bar (phosphinothricin acetyltransferase), ble (bleomycin binding protein), cat (chloramphenicol acetyltransferase), hygB (hygromycin B phosphotransferase), nat (nourseothricin acetyltransferase), niaD (nitrate reductase), neo (neomycin phosphotransferase), pac (puromycin acetyltransferase), pyrG (orotidine-5′-phosphate decarboxylase), sat (streptothricin acetyltransferase), sC (sulfate adenyltransferase), trpC (anthranilate synthase), and glyphosate resistant EPSPS genes. Furthermore, selection may be accomplished by co-transformation, e.g., as described in WO 91/17243, herein incorporated by reference in its entirety.

A nucleic acid sequence of the present invention may be operably linked to a suitable promoter sequence. The promoter sequence is a nucleic acid sequence which is recognized by the algal host cell for expression of the nucleic acid sequence. The promoter sequence contains transcription and translation control sequences which mediate the expression of the protein or fragment thereof.

A promoter may be any nucleic acid sequence which shows transcriptional activity in the algal host cell of choice and may be obtained from genes encoding polypeptides either homologous or heterologous to the host cell. Examples of suitable promoters for directing the transcription of a nucleic acid construct of the invention in an algal host are light harvesting protein promoters obtained from photosynthetic organisms, Chlorella virus methyltransferase promoters, CaMV 35 S promoter, PL promoter from bacteriophage λ, nopaline synthase promoter from the Ti plasmid of Agrobacterium tumefaciens, and bacterial trp promotor.

A protein or fragment thereof encoding nucleic acid molecule of the present invention may also be operably linked to a terminator sequence at its 3′ terminus. The terminator sequence may be native to the nucleic acid sequence encoding the protein or fragment thereof or may be obtained from foreign sources. Any terminator which is functional in the algal host cell of choice may be used in the present invention.

A protein or fragment thereof encoding nucleic acid molecule of the present invention may also be operably linked to a suitable leader sequence. A leader sequence is a nontranslated region of a mRNA which is important for translation by the algal host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence which is functional in the algal host cell of choice may be used in the present invention.

A polyadenylation sequence may also be operably linked to the 3′ terminus of the nucleic acid sequence of the present invention. The polyadenylation sequence is a sequence which when transcribed is recognized by the algal host to add polyadenosine residues to transcribed mRNA. The polyadenylation sequence may be native to the nucleic acid sequence encoding the protein or fragment thereof or may be obtained from foreign sources. Any polyadenylation sequence which is functional in the algal host of choice may be used in the present invention.

To avoid the necessity of disrupting the cell to obtain the protein or fragment thereof, and to minimize the amount of possible degradation of the expressed protein or fragment thereof within the cell, it is preferred that expression of the protein or fragment thereof gives rise to a product secreted outside the cell. To this end, the protein or fragment thereof of the present invention may be linked to a signal peptide linked to the amino terminus of the protein or fragment thereof. A signal peptide is an amino acid sequence which permits the secretion of the protein or fragment thereof from the algal host into the culture medium. The signal peptide may be native to the protein or fragment thereof of the invention or may be obtained from foreign sources. The 5′ end of the coding sequence of the nucleic acid sequence of the present invention may inherently contain a signal peptide coding region naturally linked in translation reading frame with the segment of the coding region which encodes the secreted protein or fragment thereof. Alternatively, the 5′ end of the coding sequence may contain a signal peptide coding region which is foreign to that portion of the coding sequence which encodes the secreted protein or fragment thereof. The foreign signal peptide may be required where the coding sequence does not normally contain a signal peptide coding region. Alternatively, the foreign signal peptide may simply replace the natural signal peptide to obtain enhanced secretion of the desired protein or fragment thereof. Any signal peptide capable of permitting secretion of the protein or fragment thereof in an algal host of choice may be used in the present invention.

A protein or fragment thereof encoding nucleic acid molecule of the present invention may also be linked to a propeptide coding region. A propeptide is an amino acid sequence found at the amino terminus of a proprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme. The propeptide coding region may be native to the protein or fragment thereof or may be obtained from foreign sources. The foreign propeptide coding region may be obtained from the Saccharomyces cerevisiae alpha-factor gene or Myceliophthora thermophila laccase gene (WO 95/33836, herein incorporated by reference in its entirety).

The procedures used to ligate the elements described above to construct the recombinant expression vector of the present invention are well known to one skilled in the art (see, for example, Sambrook, 2nd ed., et al., Molecular Cloning, A Laboratory Manual Cold Spring Harbor, N.Y., (1989), herein incorporated by reference in its entirety).

The present invention also relates to recombinant algal host cells produced by the methods of the present invention which are advantageously used with the recombinant vector of the present invention. The cell is preferably transformed with a vector comprising a nucleic acid sequence of the invention followed by integration of the vector into the host chromosome. The choice of algal host cells will to a large extent depend upon the gene encoding the protein or fragment thereof and its source.

Algal cells may be transformed by a variety of known techniques, including but not limit to, microprojectile bombardment, protoplast fusion, electroporation, microinjection, and vigorous agitation in the presence of glass beads. Suitable procedures for transformation of green algal host cells are described in EP 108 580, herein incorporated by reference in its entirety. A suitable method of transforming Chlorella species is described by Jarvis and Brown, Curr. Genet. 19: 317-321 (1991), herein incorporated by reference in its entirety. A suitable method of transforming cells of diatom Phaeodactylum tricornutum species is described in WO 97/39106, herein incorporated by reference in its entirety. Chlorophyll C-containing algae may be transformed using the procedures described in U.S. Pat. No. 5,661,017, herein incorporated by reference in its entirety.

The expressed protein or fragment thereof may be detected using methods known in the art that are specific for the particular protein or fragment. These detection methods may include the use of specific antibodies, formation of an enzyme product, or disappearance of an enzyme substrate. For example, if the protein or fragment thereof has enzymatic activity, an enzyme assay may be used. Alternatively, if polyclonal or monoclonal antibodies specific to the protein or fragment thereof are available, immunoassays may be employed using the antibodies to the protein or fragment thereof. The techniques of enzyme assay and immunoassay are well known to those skilled in the art.

The resulting protein or fragment thereof may be recovered by methods known in the arts. For example, the protein or fragment thereof may be recovered from the nutrient medium by conventional procedures including, but not limited to, centrifugation, filtration, extraction, spray-drying, evaporation, or precipitation. The recovered protein or fragment thereof may then be further purified by a variety of chromatographic procedures, e.g., ion exchange chromatography, gel filtration chromatography, affinity chromatography, or the like.

(e) Plant Constructs and Plant Transformants

Nucleic acid molecules of the present invention may be used in plant transformation or transfection. Exogenous genetic material may be transferred into a plant cell and the plant cell regenerated into a whole, fertile or sterile plant. Exogenous genetic material is any genetic material, whether naturally occurring or otherwise, from any source that is capable of being inserted into any organism. Such genetic material may be transferred into either monocotyledons and dicotyledons including but not limited to the plants, alfalfa, Arabidopsis, barley, Brassica, broccoli, cabbage, citrus, cotton, garlic, oat, oilseed rape, onion, canola, flax, maize, an ornamental plant, pea, peanut, pepper, potato, rice, rye, sorghum, soybean, strawberry, sugarcane, sugarbeet, tomato, wheat, poplar, pine, fir, eucalyptus, apple, lettuce, lentils, grape, banana, tea, turf grasses, sunflower, oil palm, Phaseolus etc. Particularly preferred plants to use for the transformation or transfection would include Arabidopsis, barley, cotton, oat, oilseed rape, rice, maize, soybean, canola, ornamentals, sugarcane, sugarbeet, tomato, potato, wheat and turf grasses (See specifically, Chistou, Particle Bombardment for Genetic Engineering of Plants, Biotechnology Intelligence Unit, Academic Press, San Diego, Calif. (1996), herein incorporated by reference in its entirety).

Transfer of a nucleic acid that encodes for a protein can result in overexpression of that protein in a transformed cell or transgenic plant One or more of the proteins or fragments thereof encoded by nucleic acid molecules of the present invention may be overexpressed in a transformed cell or transformed plant. Such overexpression may be the result of transient or stable transfer of the exogenous material. In a preferred embodiment of the present invention, one or more of the C. vulgaris homologue proteins or fragments is overexpressed in a transformed cell or transgenic plant.

Exogenous genetic material may be transferred into a plant cell by the use of a DNA vector or construct designed for such a purpose. Vectors have been engineered for transformation of large DNA inserts into plant genomes. Binary bacterial artificial chromosomes have been designed to replicate in both E. coli and A. tumefaciens and have all of the features required for transferring large inserts of DNA into plant chromosomes (Choi and Wing, http://genome.clemson.edu/protocols2-nj.html July, 1998). ApBACwich system has been developed to achieve site-directed integration of DNA into the genome. A 150 kb cotton BAC DNA is reported to have been transferred into a specific lox site in tobacco by biolistic bombardment and Cre-lox site specific recombination.

A construct or vector may also include a plant promoter to express the protein or protein fragment of choice. A number of promoters which are active in plant cells have been described in the literature. These include the nopaline synthase (NOS) promoter (Ebert et al., Proc. Natl. Acad. Sci. U.S.A. 84: 5745-5749 (1987), herein incorporated by reference in its entirety), the octopine synthase (OCS) promoter (which are carried on tumor-inducing plasmids of Agrobacterium tumefaciens), the caulimovirus promoters such as the cauliflower mosaic virus (CaMV) 19S promoter (Lawton et al., Plant Mol. Biol. 9:3 15-324 (1987), herein incorporated by reference in its entirety) and the CAMV 35S promoter (Odell et al., Nature 313: 810-812 (1985), herein incorporated by reference in its entirety), the figwort mosaic virus 35S-promoter, the light-inducible promoter from the small subunit of ribulose-1,5-bis-phosphate carboxylase (ssRUBISCO), the Adh promoter (Walker et al., Proc. Natl. Acad. Sci. U.S.A. 84: 6624-6628 (1987), herein incorporated by reference in its entirety), the sucrose synthase promoter (Yang et al., Proc. Natl. Acad. Sci. U.S.A. 87: 4144-4148 (1990), herein incorporated by reference in its entirety), the R gene complex promoter (Chandler et al., The Plant Cell 1: 1175-1183 (1989), herein incorporated by reference in its entirety), and the chlorophyll a/b binding protein gene promoter, etc. These promoters have been used to create DNA constructs which have been expressed in plants; see, e.g., PCT publication WO 84/02913, herein incorporated by reference in its entirety.

Promoters which are known or are found to cause transcription of DNA in plant cells can be used in the present invention. Such promoters may be obtained from a variety of sources such as plants and plant viruses. It is preferred that the particular promoter selected should be capable of causing sufficient expression to result in the production of an effective amount of protein to cause the desired phenotype. In addition to promoters which are known to cause transcription of DNA in plant cells, other promoters may be identified for use in the current invention by screening a plant cDNA library for genes which are selectively or preferably expressed in the target tissues or cells.

For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea (Edwards et al., Proc. Natl. Acad Sc. USA. 87: 3459-3463 (1990), herein incorporated by reference in its entirety), the chloroplast fructose-1,6-biphosphatase (FBPase) promoter from wheat (Lloyd et al., Mol. Gen. Genet. 225: 209-216 (1991), herein incorporated by reference in its entirety), the nuclear photosynthetic ST-LS1 promoter from potato (Stockhaus et al., EMBO J 8: 2445-2451 (1989), herein incorporated by reference in its entirety), the phenylalanine ammonia-lyase (PAL) promoter and the chalcone synthase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine (Yamamoto et al., Plant Cell Physiol. 35: 773-778 (1994), herein incorporated by reference in its entirety), the promoter for the Cab-1 gene from wheat (Fejes et al., Plant Mol. Biol. 15: 921-932 (1990), herein incorporated by reference in its entirety), the promoter for the CAB-1 gene from spinach (Lubberstedt et al., Plant Physiol. 104: 97-1006 (1994), herein incorporated by reference in its entirety), the promoter for the cab1 R gene from rice (Luan et al., Plant Cell. 4: 971-981 (1992), herein incorporated by reference in its entirety), the pyruvate, orthophosphate dikinase (PPDK) promoter from Zea mays (Matsuoka et al., Proc. Natl. Acad. Sci. USA. 90: 9586-9590 (1993), herein incorporated by reference in its entirety), the promoter for the tobacco Lhcb1*2 gene (Cerdan et al., Plant Mol. Biol. 33: 245-255. (1997), herein incorporated by reference in its entirety), the Arabidopsis thaliana SUC2 sucrose-H+ symporter promoter (Truernit et al., Planta. 196: 564-570 (1995), herein incorporated by reference in its entirety), and the promoter for the thylacoid membrane proteins from spinach (psaD, psaF, psaE, PC, FNR, atpC, atpD, cab, rbcS). Other promoters for the chlorophyl a/b-binding proteins may also be utilized in the present invention, such as the promoters for LhcB gene and PsbP gene from white mustard (Sinapis alba; Kretsch et al., Plant Mol. Biol. 28: 219-229 (1995), herein incorporated by reference in its entirety).

For the purpose of expression in sink tissues of the plant, such as the tuber of the potato plant, the fruit of tomato, or the seed of Zea mays, wheat, rice, and barley, it is preferred that the promoters utilized in the present invention have relatively high expression in these specific tissues. A number of promoters for genes with tuber-specific or -enhanced expression are known, including the class I patatin promoter (Bevan et al., EMBO J. 8: 1899-1906 (1986); Jefferson et al., Plant Mol. Biol. 14: 995-1006 (1990), both of which are herein incorporated by reference in its entirety), the promoter for the potato tuber ADPGPP genes, both the large and small subunits, the sucrose synthase promoter (Salanoubat and Belliard, Gene. 60: 47-56 (1987), Salanoubat and Belliard, Gene. 84: 181-185 (1989), both of which are herein incorporated by reference in their entirety), the promoter for the major tuber proteins including the 22 kd protein complexes and proteinase inhibitors (Hannapel, Plant Physiol. 101: 703-704 (1993), herein incorporated by reference in its entirety), the promoter for the granule bound starch synthase gene (GBSS) (Visser et al., Plant Mol. Biol. 17: 691-699 (1991), herein incorporated by reference in its entirety), and other class I and II patatins promoters (Koster-Topfer et al., Mol. Gen. Genet. 219: 390-396 (1989); Mignery et al., Gene. 62: 27-44 (1988), both of which are herein incorporated by reference in their entirety).

Other promoters can also be used to express a fructose 1,6 bisphosphate aldolase gene in specific tissues, such as seeds or fruits. The promoter for β-conglycinin (Chen et al, Dev. Genet. 10: 112-122 (1989), herein incorporated by reference in its entirety) or other seed-specific promoters such as the napin and phaseolin promoters, can be used. The zeins are a group of storage proteins found in Zea mays endosperm. Genomic clones for zein genes have been isolated (Pedersen et al., Cell 29: 1015-1026 (1982), herein incorporated by reference in its entirety), and the promoters from these clones, including the 15 kD, 16 kD, 19 kD, 22 kD, 27 kD, and gamma genes, could also be used. Other promoters known to function, for example, in Zea mays, include the promoters for the following genes: waxy, Brittle, Shrunken 2, Branching enzymes I and II, starch synthases, debranching enzymes, oleosins, glutelins, and sucrose synthases. A particularly preferred promoter for Zea mays endosperm expression is the promoter for the glutelin gene from rice, more particularly the Osgt-1 promoter (Zheng et al., Mol. Cell. Biol. 13: 5829-5842 (1993), herein incorporated by reference in its entirety). Examples of promoters suitable for expression in wheat include those promoters for the ADPglucose pyrophosphorylase (ADPGPP) subunits, the granule bound and other starch synthases, the branching and debranching enzymes, the embryogenesis-abundant proteins, the gliadins, and the glutenins. Examples of such promoters in rice include those promoters for the ADPGPP subunits, the granule bound and other starch synthases, the branching enzymes, the debranching enzymes, sucrose syntheses, and the glutelins. A particularly preferred promoter is the promoter for rice glutelin, Osgt-1. Examples of such promoters for barley include those for the ADPGPP subunits, the granule bound and other starch synthases, the branching enzymes, the debranching enzymes, sucrose synthases, the hordeins, the embryo globulins, and the aleurone specific proteins.

Root specific promoters may also be used. An example of such a promoter is the promoter for the acid chitinase gene (Samac et al., Plant Mol. Biol. 25: 587-596 (1994), herein incorporated by reference in its entirety). Expression in root tissue could also be accomplished by utilizing the root specific subdomains of the CaMV35S promoter that have been identified (Lam et al., Proc. Natl. Acad. Sci. USA. 86: 7890-7894 (1989), herein incorporated by reference in its entirety). Other root cell specific promoters include those reported by Conkling et al. (Conkling et al., Plant Physiol. 93: 1203-1211 (1990), herein incorporated by reference in its entirety).

Additional promoters that may be utilized are described, for example, in U.S. Pat. Nos. 5,378,619, 5,391,725, 5,428,147, 5,447,858, 5,608,144, 5,608,144, 5,614,399, 5,633,441, 5,633,435, and 4,633,436, all of which are herein incorporated by reference in their entirety. In addition, a tissue specific enhancer may be used (Fromm et al., The Plant Cell 1: 977-984 (1989), herein incorporated by reference in its entirety). It is further understood that one or more of the promoters of the present invention may be used.

Constructs or vectors may also include, with the coding region of interest, a nucleic acid sequence that acts, in whole or in part, to terminate transcription of that region. For example, such sequences have been isolated including the Tr7 3′ sequence and the nos 3′ sequence (Ingelbrecht et al., The Plant Cell 1: 671-680 (1989); Bevan et al., Nucleic Acids Res. 11: 369-385 (983), both of which are herein incorporated by reference in their entirety), or the like. It is understood that one or more sequences of the present invention that act, to terminate transcription may be used.

A vector or construct may also include other regulatory elements. Examples of such include the Adh intron 1 (Callis et al., Genes and Develop. 1: 1183-1200 (1987), herein incorporated by reference in its entirety), the sucrose synthase intron (Vasil et al., Plant Physiol. 91: 1575-1579 (1989), herein incorporated by reference in its entirety) and the TMV omega element (Gallie et al., The Plant Cell 1: 301-311 (1989), herein incorporated by reference in its entirety). These and other regulatory elements may be included when appropriate. It is also understood that one or more of the regulatory regions of the present invention may be used.

A vector or construct may also include a selectable marker. Selectable markers may also be used to select for plants or plant cells that contain the exogenous genetic material. Examples of such include, but are not limited to, a neo gene (Potrykus et al., Mol. Gen. Genet. 199: 183-188 (1985), herein incorporated by reference in its entirety) which codes for kanamycin resistance and can be selected for using kanamycin, G418, etc.; a bar gene which codes for bialaphos resistance; a mutant EPSP synthase gene (Hinchee et al., Bio/Technology 6: 915-922 (1988), herein incorporated by reference in its entirety) which encodes glyphosate resistance; a nitrilase gene which confers resistance to bromoxynil (Stalker et al., J. Biol. Chem. 263: 6310-6314 (1988), herein incorporated by reference in its entirety); a mutant acetolactate synthase gene (ALS) which confers imidazolinone or sulphonylurea resistance (European Patent Application 154,204 (Sep. 11, 1985), herein incorporated by reference in its entirety); and a methotrexate resistant DHFR gene (Thillet et al., J. Biol. Chem. 263: 12500-12508 (1988), herein incorporated by reference in its entirety).

A vector or construct may also include a transit peptide. Incorporation of a suitable chloroplast transit peptide may also be employed (European Patent Application Publication Number 0218571, herein incorporated by reference in its entirety). Translational enhancers may also be incorporated as part of the vector DNA. DNA constructs could contain one or more 5′ non-translated leader sequences which may serve to enhance expression of the gene products from the resulting mRNA transcripts. Such sequences may be derived from the promoter selected to express the gene or can be specifically modified to increase translation of the mRNA. Such regions may also be obtained from viral RNAs, from suitable eukaryotic genes, or from a synthetic gene sequence. For a review of optimizing expression of transgenes, see Koziel et al., Plant Mol. Biol. 32: 393405 (1996), herein incorporated by reference in its entirety.

A vector or construct may also include a screenable marker. Screenable markers may be used to monitor expression. Exemplary screenable markers include a O-glucuronidase or uidA gene (GUS) which encodes an enzyme for which various chromogenic substrates are known (Jefferson, Plant Mol. Biol, Rep. 5: 387405 (1987); Jefferson et al., EMBO J. 6: 3901-3907 (1987), both of which are herein incorporated by reference in their entirety); an R-locus gene, which encodes a product that regulates the production of anthocyanin pigments (red color) in plant tissues ((Dellaporta et al., Stadler Symposium 11: 263-282 (1988), herein incorporated by reference in its entirety); a β-lactamase gene (Sutcliffe et al., Proc. Natl. Acad. Sci. U.S.A. 75: 3737-3741 (1978), herein incorporated by reference in its entirety), a gene which encodes an enzyme for which various chromogenic substrates are known (e.g., PADAC, a chromogenic cephalosporin); a luciferase gene (Ow et al., Science 234: 856-859 (1986), herein incorporated by reference in its entirety) a xylE gene (Zukowsky et al., Proc. Natl. Acad. Sci. U.S.A. 80: 1101-1105 (1983), herein incorporated by reference in its entirety) which encodes a catechol diozygenase that can convert chromogenic catechols; an α-amylase gene (Ikatu et al., Bio/Technol. 8: 241-242 (1990), herein incorporated by reference in its entirety); a tyrosinase gene (Katz et al., J. Gen. Microbiol. 129: 2703-2714 (1983), herein incorporated by reference in its entirety) which encodes an enzyme capable of oxidizing tyrosine to DOPA and dopaquinone which in turn condenses to melanin; an α-galactosidase, which will turn a chromogenic α-galactose substrate.

Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins detectable, e.g., by ELISA, small active enzymes detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

There are many methods for introducing nucleic acid molecules into plant cells. Suitable methods are believed to include virtually any method by which nucleic acid molecules may be introduced into a cell, such as by Agrobacterium infection or direct delivery of nucleic acid molecules such as, for example, by PEG-mediated transformation, by electroporation or by acceleration of DNA coated particles, etc. (Potrykus, Ann. Rev. Plant Physiol. Plant Mol. Biol. 42: 205-225 (1991); Vasil, Plant Mol. Biol. 25: 925-937 (1994), both of which are herein incorporated by reference in their entirety). For example, electroporation has been used to transform Zea mays protoplasts (Fromm et al., Nature 312: 791-793 (1986), herein incorporated by reference in its entirety).

Other vector systems suitable for introducing transforming DNA into a host plant cell includes but is not limited to binary artificial chromosome (BIBAC) vectors (Hamilton et al., Gene 200:107-116, (1997), herein incorporated by reference in its entirety, and transfection with RNA viral vectors (Della-Cioppa et al., Ann. N.Y. Acad. Sci. (1996), 792 (Engineering Plants for Commercial Products and Applications), 57-61, herein incorporated by reference in its entirety.

Technology for introduction of DNA into cells is well known to those of skill in the art. Four general methods for delivering a gene into cells have been described: (1) chemical methods (Graham and van der Eb, Virology, 54: 536-539 (1973), herein incorporated by reference in its entirety); (2) physical methods such as microinjection (Capecchi, Cell 22: 479-488 (1980), herein incorporated by reference in its entirety), electroporation (Wong and Neumann, Biochem. Biophys. Res. Commun. 107: 584-587 (1982); Fromm et al., Proc. Natl. Acad. Sci. USA. 82: 5824-5828 (1985); U.S. Pat. No. 5,384,253, all of which are herein incorporated by reference in their entirety), and the gene gun (Johnston and Tang, Methods Cell Biol. 43: 353-365 (1994), herein incorporated by reference in its entirety); (3) viral vectors (Clapp, Clin. Perinatol. 20: 155-168 (1993); Lu et al., J. Exp. Med. 178: 2089-2096 (1993); Eglitis and Anderson, Biotechnique 6: 608-614 (1988), all of which the entirety are herein incorporated by reference); and (4) receptor-mediated mechanisms (Curiel et al., Hum. Gen. Ther. 3: 147-154 (1992); Wagner et al., Proc. Natl. Acad. Sci. U.S.A. 89: 6099-6103 (1992), all of which the entirety are herein incorporated by reference).

Acceleration methods that may be used include, for example, microprojectile bombardment and the like. One example of a method for delivering transforming nucleic acid molecules to plant cells is microprojectile bombardment. This method has been reviewed by Yang and Christou, eds., Particle Bombardment Technology for Gene Transfer, Oxford Press, Oxford, England (1994), herein incorporated by reference in its entirety). Non-biological particles (microprojectiles) that may be coated with nucleic acids and delivered into cells by a propelling force. Exemplary particles include those comprised of tungsten, gold, platinum, and the like.

A particular advantage of microprojectile bombardment, in addition to it being an effective means of reproducibly, and stably transforming monocotyledons, is that neither the isolation of protoplasts (Cristou et al., Plant Physiol. 87: 671-674 (1988), herein incorporated by reference in its entirety) nor the susceptibility of Agrobacterium infection is required. An illustrative embodiment of a method for delivering DNA into maize cells by acceleration is a biolistics-particle delivery system, which can be used to propel particles coated with DNA through a screen, such as a stainless steel or Nytex screen, onto a filter surface covered with corn cells cultured in suspension. Gordon-Kamm et al., describes the basic procedure for coating tungsten particles with DNA (Gordon-Kamm et al., Plant Cell 2: 603-618 (1990), herein incorporated by reference in its entirety). The screen disperses the tungsten nucleic acid particles so that they are not delivered to the recipient cells in large aggregates. A particle delivery system suitable for use with the present invention is the helium acceleration PDS-1000/He gun which is available from Bio-Rad Laboratories (Bio-Rad, Hercules, Calif.) (Sanford et al., Technique 3: 3-16 (1991), herein incorporated by reference in its entirety).

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively; immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus which express the exogenous gene product 48 hours post-bombardment often range from one to ten and average one to three.

In another alternative embodiment, plastids can be stably transformed. Methods suitable for plastid transformation in higher plants include particle gun delivery of DNA containing a selectable marker and targeting of the DNA to the plastid genome through homologous recombination (Svab et al. Proc. Natl. Acad. Sci. (U.S.A.) 87:8526-8530 (1990): Svab and Maliga Proc. Natl. Acad. Sci. (U.S.A.) 90:913-917 (1993); Staub and Maliga, P. EMBO J. 12:601-606 (1993), U.S. Pat. Nos. 5,451,513 and 5,545,818, all of which are herein incorporated by reference in their entirety).

In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described (Fraley et al., Biotechnology 3: 629-635 (1985); Rogers et al., Meth Enzymol 153: 253-277 (1987), both of which are herein incorporated by reference in their entirety. Further, the integration of the Ti-DNA is a relatively precise process resulting in few rearrangements. The region of DNA to be transferred is defined by the border sequences, and intervening DNA is usually inserted into the plant genome as described (Spielmann et al., Mol. Gen. Genet. 205: 34 (1986), herein incorporated by reference in its entirety).

Modern Agrobacterium transformation vectors are capable of replication in E. coli as well as Agrobacterium, allowing for convenient manipulations as described (Klee et al., In: Plant DNA Infectious Agents, T. Hohn and J. Schell, eds., Springer-Verlag, New York, pp. 179-203 (1985), herein incorporated by reference in its entirety). Moreover, recent technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes (Rogers et al., Meth. In Enzymol, 153: 253-277 (1987), herein incorporated by reference in its entirety). In addition, Agrobacterium containing both armed and disarmed Ti genes can be used for the transformations. In those plant strains where Agrobacterium-mediated transformation is efficient, it is the method of choice because of the facile and defined nature of the gene transfer.

A transgenic plant formed using Agrobacterium transformation methods typically contains a single gene on one chromosome. Such transgenic plants can be referred to as being heterozygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent sergeant transgenic plant that contains a single added gene, germinating some of the seed produced and analyzing the resulting plants produced for the gene of interest.

It is also to be understood that two different transgenic plants can also be mated to produce offspring that contain two independently segregating added, exogenous genes. Selfing of appropriate progeny can produce plants that are homozygous for both added, exogenous genes that encoding a polypeptide of interest. Back-crossing to a parental plant and out-crossing with a non-transgenic plant are also contemplated, as is vegetative propagation.

The present invention also provides for parts of the plants of the present invention. Plant parts, without limitation, include seed, endosperm, ovule and pollen. In a particularly preferred embodiment of the present invention, the plant part is a seed.

Transformation of plant protoplasts can be achieved using methods based on calcium phosphate precipitation, polyethylene glycol treatment, electroporation, and combinations of these treatments. See for example (Potrykus et al., Mol. Gen. Genet. 205: 193-200 (1986); Lorz et al, Mol. Gen. Genet. 199: 178, (1985); Fromm et al, Nature 319: 791 (1986); Uchimiya et al., Mol. Gem Genet. 204:204 (1986); Callis et al., Genes and Development 1183 (1987); Marcotte et al., Nature 335:454 (1988), all of which are herein incorporated by reference in their entirety).

Application of these systems to different plant strains depends upon the ability to regenerate that particular plant strain from protoplasts. Illustrative methods for the regeneration of cereals from protoplasts are described (Fujimura et al., Plant Tissue Culture Letters 2: 74 (1985); Toriyama et al, Theor Appl. Genet. 205: 34 (1986); Yamada et al, Plant Cell Rep. 4: 85 (1986); Abdullah et al., Biotechnology 4: 1087 (1986), all of which are herein incorporated by reference in their entirety).

To transform plant strains that cannot be successfully regenerated from protoplasts, other ways to introduce DNA into intact cells or tissues can be utilized. For example, regeneration of cereals from immature embryos or explants can be effected as described (Vasil, Biotechnology 6: 397 (1988), herein incorporated by reference in its entirety). In addition, “particle gun” or high-velocity microprojectile technology can be utilized (Vasil et al., Bio/Technology 10: 667, (1992), herein incorporated by reference in its entirety).

Using the latter technology, DNA is carried through the cell wall and into the cytoplasm on the surface of small metal particles as described (Klein et al., Nature 328: 70 (1987); Klein et al., Proc. Natl. Acad Sci U.S.A. 85: 8502-8505 (1988); McCabe et al., Biotechnology 6:923 (1988), all of which are herein incorporated by reference in their entirety). The metal particles penetrate through several layers of cells and thus allow the transformation of cells within tissue explants.

Other methods of cell transformation can also be used and include but are not limited to introduction of DNA into plants by direct DNA transfer into pollen (Zhou et al., Mete Enzymol. 101: 433 (1983); Hess et al., Intern Rev. Cytol. 107:367 (1987); Luo et al, Plant Mol. Biol. Reporter 6: 165 (1988), all of which are herein incorporated by reference in their entirety), by direct injection of DNA into reproductive organs of a plant (Pena et al., Nature 325: 274 (1987), herein incorporated by reference in its entirety), or by direct injection of DNA into the cells of immature embryos followed by the rehydration of desiccated embryos (Neuhaus et al., Theor. Appl. Genet. 75: 30, (1987), herein incorporated by reference in its entirety).

The regeneration, development, and cultivation of plants from single plant protoplast transformants or from various transformed explants is well known in the art (Weissbach and Weissbach, In: Methods for Plant Molecular Biology, (Eds.), Academic Press, Inc. San Diego, Calif., (1988), herein incorporated by reference in its entirety). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants, as discussed before. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908, all of which are herein incorporated by reference in their entirety); soybean (U.S. Pat. No. 5,569,834, U.S. Pat. No. 5,416,011, McCabe et al., Biotechnology 6: 923 (1988), Christou et al., Plant Physiol. 87: 671-674 (1988), all of which are herein incorporated by reference in their entirety); Brassica (U.S. Pat. No. 5,463,174, herein incorporated by reference in its entirety); peanut (Cheng et al., Plant Cell Rep. 15: 653-657 (1996), McKently et al., Plant Cell Rep. 14: 699-703 (1995), all of which are herein incorporated by reference in their entirety); papaya (Yang et al., (1996), herein incorporated by reference in its entirety); pea (Grant et al., Plant Cell Rep. 15: 254-258, (1995), herein incorporated by reference in its entirety).

Transformation of monocotyledons using electroporation, particle bombardment, and Agrobacterium have also been reported. Transformation and plant regeneration have been achieved in asparagus (Bytebier et al., Proc. Natl. Acad. Sci. USA. 84: 5345, (1987), herein incorporated by reference in its entirety); barley (Wan and Lemaux, Plant Physiol 104: 37 (1994), herein incorporated by reference in its entirety); maize (Rhodes et al., Science 240: 204 (1988), Gordon-Kamm et al., Plant Cell 2: 603, (1990), Fromm et al., Bio/Technology 8: 833 (1990), Koziel et al., Bio/Technology 11: 194 (1993), Armstrong et al., Crop Science 35: 550-557 (1995), all of which are herein incorporated by reference in their entirety); oat (Somers et al., Bio/Technology 10: 1589 (1992), herein incorporated by reference in its entirety); orchardgrass (Horn et al., Plant Cell Rep. 7: 469 (1988), herein incorporated by reference in its entirety); rice (Toriyama et al., Theor Appl. Genet. 205: 34 (1986); Park et al., Plant Mol. Biol. 32: 1135-1148, (1996); Abedinia et al., Aust. J. Plant Physiol. 24:133-141, (1997); Zhang and Wu, Theor. Appl. Genet. 76: 835, (1988); Thang et al. Plant Cell Rep. 7: 379, (1988); Battraw and Hall, Plant Sci. 86: 191-202, (1992); Christou et al., Bio/Technology 9: 957, (1991), all of which are herein incorporated by reference in their entirety); sugarcane (Bower and Birch, Plant J 2: 409, (1992), herein incorporated by reference in its entirety); tall fescue (Wang et al., Bio/Technology 10:691 (1992), herein incorporated by reference in its entirety), and wheat (Vasil et al., Bio/Technology 10:667 (1992); U.S. Pat. No. 5,631,152, both of which are herein incorporated by reference in their entirety.

Assays for gene expression based on the transient expression of cloned nucleic acid constructs have been developed by introducing the nucleic acid molecules into plant cells by polyethylene glycol treatment, electroporation, or particle bombardment (Marcotte et al., Nature 335: 454-457 (1988); Marcotte et al., Plant Cell 1: 523-532 (1989); McCarty et al., Cell 66: 895-905 (1991); Hattori et al., Genes Dev. 6: 609-618 (1992); Goff et al., EMBO J. 9: 2517-2522 (1990), all of which are herein incorporated by reference in their entirety). Transient expression systems may be used to functionally dissect gene constructs (See generally, Mailga et al., Methods in Plant Molecular Biology, Cold Spring Harbor Press (1995), herein incorporated by reference in its entirety).

Any of the nucleic acid molecules of the present invention may be introduced into a plant cell in a permanent or transient manner in combination with other genetic elements such as vectors, promoters enhancers etc. Further any of the C. vulgaris gene homologue or fragment thereof homologies of the present invention may be introduced into a plant cell in a manner that allows for over expression of the protein or fragment thereof encoded by the nucleic acid molecule.

Antibodies have been expressed in plants (Hiatt et al., Nature 342: 76-78 (1989); Conrad and Fielder, Plant Mol. Biol. 26: 1023-1030 (1994), both of which are herein incorporated by reference in their entirety). Cytoplamsic expression of a scFv (single-chain Fv antibodies) has been reported to delay infection by artichoke mottled crinkle virus. Transgenic plants that express antibodies directed against endogenous proteins may exhibit a physiological effect (Philips et al., EMBO J. 16:4489-4496 (1997); Marion-Poll, Trends in Plant Science 2:447-448 (1997), both of which are herein incorporated by reference in their entirety). For example, expressed anti-abscisic antibodies reportedly result in a general perturbation of seed development (Philips et al., EMBO J. 16:4489-4496 (1997), herein incorporated by reference in its entirety).

Antibodies that are catalytic may also be expressed in plants (abzymes). The principle behind abzymes is that since antibodies may be raised against many molecules, this recognition ability can be directed toward generating antibodies that bind transition states to force a chemical reaction forward (Persidas, Nature Biotechnology 15: 1313-1315 (1997); Baca et al., Ann. Rev. Biophys. Biomol. Struct. 26: 461-493 (1997), both of which are herein incorporated by reference in their entirety). The catalytic abilities of abzymes may be enhanced by site directed mutagensis. Examples of abzymes are, for example, set forth in U.S. Pat. No. 5,658,753; U.S. Pat. No. 5,632,990; U.S. Pat. No. 5,631,137; U.S. Pat. No. 5,602,015; U.S. Pat. No. 5,559,538; U.S. Pat. No. 5,576,174; U.S. Pat. No. 5,500,358; U.S. Pat. No. 5,318,897; U.S. Pat. No. 5,298,409; U.S. Pat. No. 5,258,289 and U.S. Pat. No. 5,194,585, all of which are herein incorporated in their entirety.

It is understood that any of the antibodies of the present invention may be expressed in plants and that such expression can result in a physiological effect. It is also understood that any of the expressed antibodies may be catalytic.

(f) Fungal Constructs and Fungal Transformants

The present invention also relates to a fungal recombinant vector comprising exogenous genetic material. The present invention also relates to a fungal cell comprising a fungal recombinant vector. The present invention also relates to methods for obtaining a recombinant fungal host cell comprising introducing into a fungal host cell exogenous genetic material.

Exogenous genetic material may be transferred into a fungal cell. Exogenous genetic material is any genetic material, whether naturally occurring or otherwise, from any source that is capable of being inserted into any organism. In a preferred embodiment the exogenous genetic material includes a nucleic acid molecule having a sequence selected from the group consisting of SEQ ID NO: 1 through SEQ ID NO: 3519 or complements thereof.

The fungal recombinant vector may be any vector which can be conveniently subjected to recombinant DNA procedures. The choice of a vector will typically depend on the compatibility of the vector with the fungal host cell into which the vector is to be introduced. The vector may be a linear or a closed circular plasmid. The vector system may be a single vector or plasmid or two or more vectors or plasmids which together contain the total DNA to be introduced into the genome of the fungal host.

The fungal vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the fungal cell, is integrated into the genome and replicated together with the chromosome(s) into which it has been integrated. For integration, the vector may rely on the nucleic acid sequence of the vector for stable integration of the vector into the genome by homologous or nonhomologous recombination. Alternatively, the vector may contain additional nucleic acid sequences for directing integration by homologous recombination into the genome of the fungal host. The additional nucleic acid sequences enable the vector to be integrated into the host cell genome at a precise location(s) in the chromosome(s). To increase the likelihood of integration at a precise location, there should be preferably two nucleic acid sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the fungal host cell, and, furthermore, may be non-encoding or encoding sequences.

For autonomous replication, the vector may further comprise an origin of replication enabling the vector to replicate autonomously in the host cell in question. Examples of origin of replications for use in a yeast host cell are the 2 micron origin of replication and the combination of CEN3 and ARS 1. Any origin of replication may be used which is compatible with the fungal host cell of choice.

The vectors of the present invention preferably contain one or more selectable markers which permit easy selection of transformed cells. A selectable marker is a gene the product of which provides, for example biocide or viral resistance, resistance to heavy metals, prototrophy to auxotrophs, and the like. The selectable marker may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), and sC (sulfate adenyltransferase), and trpC (anthranilate synthase). Preferred for use in an Aspergillus cell are the amdS and pyrG markers of Aspergillus nidulans or Aspergillus oryzae and the bar marker of Streptomyces hygroscopicus. Furthermore, selection may be accomplished by cotransformation, e.g., as described in WO 91/17243, herein incorporated by reference in its entirety. A nucleic acid sequence of the present invention may be operably linked to a suitable promoter sequence. The promoter sequence is a nucleic acid sequence which is recognized by the fungal host cell for expression of the nucleic acid sequence. The promoter sequence contains transcription and translation control sequences which mediate the expression of the protein or fragment thereof.

A promoter may be any nucleic acid sequence which shows transcriptional activity in the fungal host cell of choice and may be obtained from genes encoding polypeptides either homologous or heterologous to the host cell. Examples of suitable promoters for directing the transcription of a nucleic acid construct of the invention in a filamentous fungal host are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and hybrids thereof. In a yeast host, a useful promoter is the Saccharomyces cerevisiae enolase (eno-1) promoter. Particularly preferred promoters are the TAKA amylase, NA2-tpi (a hybrid of the promoters from the genes encoding Aspergillus niger neutral alpha-amylase and Aspergillus oryzae triose phosphate isomerase), and glaA promoters.

A protein or fragment thereof encoding nucleic acid molecule of the present invention may also be operably linked to a terminator sequence at its 3′ terminus. The terminator sequence may be native to the nucleic acid sequence encoding the protein or fragment thereof or may be obtained from foreign sources. Any terminator which is functional in the fungal host cell of choice may be used in the present invention, but particularly preferred terminators are obtained from the genes encoding Aspergillus oryzae TAKA amylase, Aspergillus niger glucoamylase, Aspergillus nidulans anthranilate synthase, Aspergillus niger alpha-glucosidase, and Saccharomyces cerevisiae enolase.

A protein or fragment thereof encoding nucleic acid molecule of the present invention may also be operably linked to a suitable leader sequence. A leader sequence is a non-translated region of a mRNA which is important for translation by the fungal host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence which is functional in the fungal host cell of choice may be used in the present invention, but particularly preferred leaders are obtained from the genes encoding Aspergillus oryzae TAKA amylase and Aspergillus oryzae triose phosphate isomerase.

A polyadenylation sequence may also be operably linked to the 3′ terminus of the nucleic acid sequence of the present invention. The polyadenylation sequence is a sequence which when transcribed is recognized by the fungal host to add polyadenosine residues to transcribed mRNA. The polyadenylation sequence may be native to the nucleic acid sequence encoding the protein or fragment thereof or may be obtained from foreign sources. Any polyadenylation sequence which is functional in the fungal host of choice may be used in the present invention, but particularly preferred polyadenylation sequences are obtained from the genes encoding Aspergillus oryzae TAKA amylase, Aspergillus niger glucoamylase, Aspergillus nidulans anthranilate synthase, and Aspergillus niger alpha-glucosidase.

To avoid the necessity of disrupting the cell to obtain the protein or fragment thereof, and to minimize the amount of possible degradation of the expressed protein or fragment thereof within the cell, it is preferred that expression of the protein or fragment thereof gives rise to a product secreted outside the cell. To this end, the protein or fragment thereof of the present invention may be linked to a signal peptide linked to the amino terminus of the protein or fragment thereof. A signal peptide is an amino acid sequence which permits the secretion of the protein or fragment thereof from the fungal host into the culture medium. The signal peptide may be native to the protein or fragment thereof of the invention or may be obtained from foreign sources. The 5′ end of the coding sequence of the nucleic acid sequence of the present invention may inherently contain a signal peptide coding region naturally linked in translation reading frame with the segment of the coding region which encodes the secreted protein or fragment thereof. Alternatively, the 5′ end of the coding-sequence may contain a signal peptide coding region which is foreign to that portion of the coding sequence which encodes the secreted protein or fragment thereof. The foreign signal peptide may be required where the coding sequence does not normally contain a signal peptide coding region. Alternatively, the foreign signal peptide may simply replace the natural signal peptide to obtain enhanced secretion of the desired protein or fragment thereof. The foreign signal peptide coding region may be obtained from a glucoamylase or an amylase gene from an Aspergillus species, a lipase or proteinase gene from Rhizomucor miehei, the gene for the alpha-factor from Saccharomyces cerevisiae, or the calf preprochymosin gene. An effective signal peptide for fungal host cells is the Aspergillus oryzae TAKA amylase signal, Aspergillus niger neutral amylase signal, the Rhizomucor miehei aspartic proteinase signal, the Humicola lanuginosus cellulase signal, or the Rhizomucor miehei lipase signal. However, any signal peptide capable of permitting secretion of the protein or fragment thereof in a fungal host of choice may be used in the present invention.

A protein or fragment thereof encoding nucleic acid molecule of the present invention may also be linked to a propeptide coding region. A propeptide is an amino acid sequence found at the amino terminus of a proprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme. The propeptide coding region may be native to the protein or fragment thereof or may be obtained from foreign sources. The foreign propeptide coding region may be obtained from the Saccharomyces cerevisiae alpha-factor gene or Myceliophthora thermophila laccase gene (WO 95/33836, herein incorporated by reference in its entirety).

The procedures used to ligate the elements described above to construct the recombinant expression vector of the present invention are well known to one skilled in the art (see, for example, Sambrook, 2nd ed., et al., Molecular Cloning, A Laboratory Manual Cold Spring Harbor, N.Y., (1989)).

The present invention also relates to recombinant fungal host cells produced by the methods of the present invention which are advantageously used with the recombinant vector of the present invention. The cell is preferably transformed with a vector comprising a nucleic acid sequence of the invention followed by integration of the vector into the host chromosome. The choice of fungal host cells will to a large extent depend upon the gene encoding the protein or fragment thereof and its source. The fungal host cell may be a yeast cell or a filamentous fungal cell.

“Yeast” as used herein includes Ascosporogenous yeast (Endomycetales), Basidiosporogenous yeast, and yeast belonging to the Fungi Imperfecti (Blastomycetes). The Ascosporogenous yeasts are divided into the families Spermophthoraceae and Saccharomycetaceae. The latter is comprised of four subfamilies, Schizosaccharonzycoideae (for example, genus Schizosaccharomyces), Nadsonioideae, Lipomycoideae, and Saccharomycoideae (for example, genera Pichia Kluyveromyces and Saccharomyces). The Basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (for example, genera Sorobolomyces and Bullera) and Cryptococcaceae (for example, genus Candida). Since the classification of yeast may change in the future, for the purposes of this invention, yeast shall be defined as described in Biology and Activities of Yeast (Skinner et al., eds, Soc. App. Bacteriol. Symposium Series No. 9, (1980), herein incorporated by reference in its entirety). The biology of yeast and manipulation of yeast genetics are well known in the art (see, for example, Biochemistry and Genetics of Yeast, Bacil, Horecker, and Stopani, editors, 2nd edition, 1987; The Yeasts, Rose, and Harrison, editors, 2nd edition, (1987); and The Molecular Biology of the Yeast Saccharomyces, Strathern et al., editors, (1981), all of which are herein incorporated by reference in their entirety).

“Fungi” as used herein includes the phyla Ascomycota; Basidiomycota, Chytridiomycota, and Zygomycota (as defined by Hawksworth et al., In: Ainsworth and Bisby's Dictionary of The Fungi, 8th edition, 1995, CAB International, University Press, Cambridge, UK; herein incorporated by reference in its entirety) as well as the Oomycota (as cited in Hawksworth et al., In: Ainsworth and Bisby's Dictionary of The Fungi, 8th edition, 1995, CAB International, University Press, Cambridge, UK) and all mitosporic fungi (Hawksworth et al., In: Ainsworth and Bisby's Dictionary of The Fungi, 8th edition, 1995, CAB International, University Press, Cambridge, UK). Representative groups of Ascomycota include, for example, Neurospora, Eupenicillium (=Penicillium), Emericella (=Aspergillus), Eurotiun (=Aspergillus), and the true yeasts listed above. Examples of Basidiomycota include mushrooms, rusts, and smuts. Representative groups of Chytridiomycota include, for example, Allomyces, Blastocladiella, Coelomomyces, and aquatic fungi. Representative groups of Oomycota include, for example, Saprolegniomycetous aquatic fungi (water molds) such as Achlya. Examples of mitosporic fungi include Aspergillus, Penicilliun, Candida, and Alternaria. Representative groups of Zygomycota include, for example, Rhizopus and Mucor.

“Filamentous fungi” include all filamentous forms of the subdivision Eumycota and Oomycota (as defined by Hawksworth et al., In: Ainsworth and Bisby's Dictionary of The Fungi, 8th edition, 1995, CAB International, University Press, Cambridge, UK). The filamentous fungi are characterized by a vegetative mycelium composed of chitin, cellulose, glucan, chitosan, mannan, and other complex polysaccharides. Vegetative growth is by hyphal elongation and carbon catabolism is obligatory aerobic. In contrast, vegetative growth by yeasts such as Saccharomyces cerevisiae is by budding of a unicellular thallus and carbon catabolism may be fermentative.

In one embodiment, the fungal host cell is a yeast cell. In a preferred embodiment, the yeast host cell is a cell of the species of Candida, Kluyveromyces, Saccharomyces, Schizosaccharomyces, Pichia, and Yarrowia In a preferred embodiment, the yeast host cell is a Saccharomyces cerevisiae cell, a Saccharomyces carlsbergensis, Saccharomyces diastaticus cell, a Saccharomyces douglasii cell, a Saccharomyces kluyveri cell, a Saccharomyces norbensis cell, or a Saccharomyces oviformis cell. In another preferred embodiment, the yeast host cell is a Kluyveromyces lactis cell. In another preferred embodiment, the yeast host cell is a Yarrowia lipolytica cell.

In another embodiment, the fungal host cell is a filamentous fungal cell. In a preferred embodiment, the filamentous fungal host cell is a cell of the species of, but not limited to, Acremonium, Aspergillus, Fusarium, Humicola, Myceliophthora, Mucor, Neurospora, Penicillium, Thielavia, Tolypocladium, and Trichoderma. In a preferred embodiment, the filamentous fungal host cell is an Aspergillus cell. In another preferred embodiment, the filamentous fungal host cell is an Acremonium cell. In another preferred embodiment, the filamentous fungal host cell is a Fusarium cell. In another preferred embodiment, the filamentous fungal host cell is a Humicola cell. In another preferred embodiment, the filamentous fungal host cell is a Myceliophthora cell. In another even preferred embodiment, the filamentous fungal host cell is a Mucor cell. In another preferred embodiment, the filamentous fungal host cell is a Neurospora cell. In another preferred embodiment, the filamentous fungal host cell is a Penicillium cell. In another preferred embodiment, the filamentous fungal host cell is a Thielavia cell. In another preferred embodiment, the filamentous fungal host cell is a Tolypocladiun cell. In another preferred embodiment, the filamentous fungal host cell is a Trichoderma cell. In a preferred embodiment, the filamentous fungal host cell is an Aspergillus oryzae cell, an Aspergillus niger cell, an Aspergillus foetidus cell, or an Aspergillus japonicus cell. In another preferred embodiment, the filamentous fungal host cell is a Fusarium oxysporum cell or a Fusarium graminearum cell. In another preferred embodiment, the filamentous fungal host cell is a Humicola insolens cell or a Humicola lanuginosus cell. In another preferred embodiment, the filamentous fungal host cell is a Myceliophthora thermophila cell. In a most preferred embodiment, the filamentous fungal host cell is a Mucor miehei cell. In a most preferred embodiment, the filamentous fungal host cell is a Neurospora crassa cell. In a most preferred embodiment, the filamentous fungal host cell is a Penicillium purpurogenum cell. In another most preferred embodiment, the filamentous fungal host cell is a Thielavia terrestris cell. In another most preferred embodiment, the Trichoderma cell is a Trichoderma reesei cell, a Trichoderma viride cell, a Trichoderma longibrachiatum cell, a Trichoderma harzianum cell, or a Trichoderma koningii cell. In a particularly preferred embodiment, the fungal host cell is selected from an A. nidulans cell, an A. niger cell, an A. oryzae cell and an A. sojae cell. In a further particularly preferred embodiment, the fungal host cell is an A. nidulans cell.

The recombinant fungal host cells of the present invention may further comprise one or more sequences which encode one or more factors that are advantageous in the expression of the protein or fragment thereof, for example, an activator (e.g., a trans-acting factor), a chaperone, and a processing protease. The nucleic acids encoding one or more of these factors are preferably not operably linked to the nucleic acid encoding the protein or fragment thereof. An activator is a protein which activates transcription of a nucleic acid sequence encoding a polypeptide (Kudla et al., EMBO 9: 1355-1364 (1990); Jarai and Buxton, Current Genetics 26: 2238-244 (1994); Verdier, Yeast 6: 271-297 (1990), all of which are herein incorporated by reference in their entirety). The nucleic acid sequence encoding an activator may be obtained from the genes encoding Saccharomyces cerevisiae heme activator protein 1 (hap1), Saccharomyces cerevisiae galactose metabolizing protein 4 (gal4), and Aspergillus nidulans ammonia regulation protein (areA). For further examples, see Verdier, Yeast 6: 271-297 (1990); MacKenzie et al., Journal of Gen. Microbiol. 139: 2295-2307 (1993), both of which are herein incorporated by reference in their entirety). A chaperone is a protein which assists another protein in folding properly (Hard et al., TIBS 19: 20-25 (1994); Bergeron et al., TIBS 19: 124-128 (1994); Demolder et al., J. Biotechnology 32: 179-189 (1994); Craig, Science 260: 1902-1903 (1993); Gething and Sambrook, Nature 355: 3345 (1992); Puig and Gilbert, J. Biol. Chem. 269: 7764-7771 (1994); Wang and Tsou, FASEB Journal 7: 1515-11157 (1993); Robinson et al, Bio/Technology 1: 381-384 (1994), all of which are herein incorporated by reference in their entirety). The nucleic acid sequence encoding a chaperone may be obtained from the genes encoding Aspergillus oryzae protein disulphide isomerase, Saccharomyces cerevisiae calnexin, Saccharomyces cerevisiae BiP/GRP78, and Saccharomyces cerevisiae Hsp70. For further examples, see Gething and Sambrook, Nature 355: 3345 (1992); Hartl et al., TIBS 19: 20-25 (1994), both of which are herein incorporated by reference in their entirety. A processing protease is a protease that cleaves a propeptide to generate a mature biochemically active polypeptide (Enderlin and Ogrydziak, Yeast 10: 67-79 (1994); Fuller et al., Proc. Natl. Acad. Sci. (U.S.A.) 86: 1434-1438 (1989); Julius et al., Cell 37: 1075-1089 (1984); Julius et al., Cell 32: 839-852 (1983), all of which are incorporated by reference in their entirety). The nucleic acid sequence encoding a processing protease may be obtained from the genes encoding Aspergillus niger Kex2, Saccharomyces cerevisiae dipeptidylaminopeptidase, Saccharomyces cerevisiae Kex2, and Yarrowia lipolytica dibasic processing endoprotease (xpr6). Any factor that is functional in the fungal host cell of choice may be used in the present invention.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., Proc. Natl. Acad. Sci. (U.S.A.) 81: 1470-1474 (1984), both of which are herein incorporated by reference in their entirety. A suitable method of transforming Fusarium species is described by Malardier et al., Gene 78: 147-156 (1989), herein incorporated by reference in its entirety. Yeast may be transformed using the procedures described by Becker and Guarente, In: Abelson and Simon, (eds.), Guide to Yeast Genetics and Molecular Biology, Methods Enzymol., Volume 194, pp 182-187, Academic Press, Inc., New York; Ito et al., J. Bacteriology 153: 163 (1983); Hinnen et al., Proc. Natl. Acad. Sci. (U.S.A.) 75: 1920, (1978), all of which are herein incorporated by reference in their entirety.

The present invention also relates to methods of producing the protein or fragment thereof comprising culturing the recombinant fungal host cells under conditions conducive for expression of the protein or fragment thereof. The fungal cells of the present invention are cultivated in a nutrient medium suitable for production of the protein or fragment thereof using methods known in the art For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the protein or fragment thereof to be expressed and/or isolated. The cultivation takes place in a suitable nutrient medium comprising carbon and nitrogen sources and inorganic salts, using-procedures known in the art (see, e.g., Bennett, and LaSure, eds., More Gene Manipulations in Fungi, Academic Press, CA, (1991), herein incorporated by reference in its entirety). Suitable media are available from commercial suppliers or may be prepared according to published compositions (e.g., in catalogues of the American Type Culture Collection, Manassas, Va.). If the protein or fragment thereof is secreted into the nutrient medium, a protein or fragment thereof can be recovered directly from the medium. If the protein or fragment thereof is not secreted, it is recovered from cell lysates.

The expressed protein or fragment thereof may be detected using methods known in the art that are specific for the particular protein or fragment These detection methods may include the use of specific antibodies, formation of an enzyme product, or disappearance of an enzyme substrate. For example, if the protein or fragment thereof has enzymatic activity, an enzyme assay may be used. Alternatively, if polyclonal or monoclonal antibodies specific to the protein or fragment thereof are available, immunoassays may be employed using the antibodies to the protein or fragment thereof. The techniques of enzyme assay and immunoassay are well known to those skilled in the art.

The resulting protein or fragment thereof may be recovered by methods known in the arts For example, the protein or fragment thereof may be recovered from the nutrient medium by conventional procedures including, but not limited to, centrifugation, filtration, extraction, spray-drying, evaporation, or precipitation. The recovered protein or fragment thereof may then be further purified by a variety of chromatographic procedures, e.g., ion exchange chromatography, gel filtration chromatography, affinity chromatography, or the like.

(g) Mammalian Constructs and Transformed Mammalian Cells

The present invention also relates to methods for obtaining a recombinant mammalian host cell, comprising introducing into a mammalian host cell exogenous genetic material. The present invention also relates to a mammalian cell comprising a mammalian recombinant vector. The present invention also relates to methods for obtaining a recombinant mammalian host cell, comprising introducing into a mammalian cell exogenous genetic material.

Mammalian cell lines available as hosts for expression are known in the art and include many immortalized cell lines available from the American Type Culture Collection (ATCC, Manassas, Va.), such as HeLa cells, Chinese hamster ovary (CHO) cells, baby hamster kidney (BHK) cells, and a number of other cell lines. Suitable promoters for mammalian cells are also known in the art and include viral promoters such as that from Simian Virus 40 (SV40) (Fiers et al., Nature 273: 113 (1978), herein incorporated by reference in its entirety), Rous sarcoma virus (RSV), adenovirus (ADV), and bovine papilloma virus (BPV). Mammalian cells may also require terminator sequences and poly-A addition sequences. Enhancer sequences which increase expression may also be included, and sequences which promote amplification of the gene may also be desirable (for example methotrexate resistance genes).

Vectors suitable for replication in mammalian cells may include viral replicons, or sequences which insure integration of the appropriate sequences encoding HCV epitopes into the host genome. For example, another vector used to express foreign DNA is vaccinia virus. In this case, for example, a nucleic acid molecule encoding an C. vulgaris protein homologue or fragment thereof is inserted into the vaccinia genome. Techniques for the insertion of foreign DNA into the vaccinia virus genome are known in the art, and may utilize, for example, homologous recombination. Such heterologous DNA is generally inserted into a gene which is non-essential to the virus, for example, the thymidine kinase gene (tk), which also provides a selectable marker. Plasmid vectors that greatly facilitate the construction of recombinant viruses have been described (see, for example, Mackett et al, J Virol. 49: 857 (1984); Chakrabarti et al., Mol. Cell. Biol. 5: 3403 (1985); Moss, In: Gene Transfer Vectors For Mammalian Cells (Miller and Calos, eds., Cold Spring Harbor Laboratory, N.Y., p. 10, (1987); all of which are herein incorporated by reference in their entirety). Expression of the HCV polypeptide then occurs in cells or animals which are infected with the live recombinant vaccinia virus.

The sequence to be integrated into the mammalian sequence may be introduced into the primary host by any convenient means, which includes calcium precipitated DNA, spheroplast fusion, transformation, electroporation, biolistics, lipofection, microinjection, or other convenient means. Where an amplifiable gene is being employed, the amplifiable gene may serve as the selection marker for selecting hosts into which the amplifiable gene has been introduced. Alternatively, one may include with the amplifiable gene another marker, such as a drug resistance marker, e.g. neomycin resistance (G418 in mammalian cells), hygromycin in resistance etc., or an auxotrophy marker (HIS3, TRP1, LEU2, URA3, ADE2, LYS2, etc.) for use in yeast cells.

Depending upon the nature of the modification and associated targeting construct, various techniques may be employed for identifying targeted integration. Conveniently, the DNA may be digested with one or more restriction enzymes and the fragments probed with an appropriate DNA fragment which will identify the properly sized restriction fragment associated with integration.

One may use different promoter sequences, enhancer sequences, or other sequence which will allow for enhanced levels of expression in the expression host. Thus, one may combine an enhancer from one source, a promoter region from another source, a 5′-noncoding region upstream from the initiation methionine from the same or different source as the other sequences, and the like. One may provide for an intron in the non-coding region with appropriate splice sites or for an alternative 3′-untranslated sequence or polyadenylation site. Depending upon the particular purpose of the modification, any of these sequences may be introduced, as desired.

Where selection is intended, the sequence to be integrated will have with it a marker gene, which allows for selection. The marker gene may conveniently be downstream from the target gene and may include resistance to a cytotoxic agent, e.g. antibiotics, heavy metals, or the like, resistance or susceptibility to HAT, gancyclovir, etc., complementation to an auxotrophic host, particularly by using an auxotrophic yeast as the host for the subject manipulations, or the like. The marker gene may also be on a separate DNA molecule, particularly with primary mammalian cells. Alternatively, one may screen the various transformants, due to the high efficiency of recombination in yeast, by using hybridization analysis, PCR, sequencing, or the like.

For homologous recombination, constructs can be prepared where the amplifiable gene will be flanked, normally on both sides with DNA homologous with the DNA of the target region. Depending upon the nature of the integrating DNA and the purpose of the integration, the homologous DNA will generally be within 100 kb, usually 50 kb, preferably about 25 kb, of the transcribed region of the target gene, more preferably within 2 kb of the target gene. Where modeling of the gene is intended, homology will usually be present proximal to the site of the mutation. By gene is intended the coding region and those sequences required for transcription of a mature mRNA. The homologous DNA may include the 5′-upstream region outside of the transcriptional regulatory region or comprising any enhancer sequences, transcriptional initiation sequences, adjacent sequences, or the like. The homologous region may include a portion of the coding region, where the coding region may be comprised only of an open reading frame or combination of exons and introns. The homologous region may comprise all or a portion of an intron, where all or a portion of one or more exons may also be present. Alternatively, the homologous region may comprise the 3′-region, so as to comprise all or a portion of the transcriptional termination region, or the region 3′ of this region. The homologous regions may extend over all or a portion of the target gene or be outside the target gene comprising all or a portion of the transcriptional regulatory regions and/or the structural gene.

The integrating constructs may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like. Usually during the preparation of a construct where various fragments are joined, the fragments, intermediate constructs and constructs will be carried on a cloning vector comprising a replication system functional in a prokaryotic host, e.g., E. coli, and a marker for selection, e.g., biocide resistance, complementation to an auxotrophic host, etc. Other functional sequences may also be present, such as polylinkers, for ease of introduction and excision of the construct or portions thereof, or the like. A large number of cloning vectors are available such as pBR322, the pUC series, etc. These constructs may then be used for integration into the primary mammalian host.

In the case of the primary mammalian host, a replicating vector may be used. Usually, such vector will have a viral replication system, such as SV40, bovine papilloma virus, adenovirus, or the like. The linear DNA sequence vector may also have a selectable marker for identifying transfected cells. Selectable markers include the neo gene, allowing for selection with G418, the herpes tk gene for selection with HAT medium, the gpt gene with mycophenolic acid, complementation of an auxotrophic host, etc.

The vector may or may not be capable of stable maintenance in the host. Where the vector is capable of stable maintenance, the cells will be screened for homologous integration of the vector into the genome of the host, where various techniques for curing the cells may be employed. Where the vector is not capable of stable maintenance, for example, where a temperature sensitive replication system is employed, one may change the temperature from the permissive temperature to the non-permissive temperature, so that the cells may be cured of the vector. In this case, only those cells having integration of the construct comprising the amplifiable gene and, when present, the selectable marker, will be able to survive selection.

Where a selectable marker is present, one may select for the presence of the targeting construct by means of the selectable marker. Where the selectable marker is not present, one may select for the presence of the construct by the amplifiable gene. For the neo gene or the herpes tk gene, one could employ a medium for growth of the transformants of about 0.1-1 mg/ml of G418 or may use HAT medium, respectively. Where DHFR is the amplifiable gene, the selective medium may include from about 0.01-0.5 mu M of methotrexate or be deficient in glycine-hypoxanthine-thymidine and have dialysed serum (GHT media).

The DNA can be introduced into the expression host by a variety of techniques that include calcium phosphate/DNA co-precipitates, microinjection of DNA into the nucleus, electroporation, yeast protoplast fusion with intact cells, transfection, polycations, e.g., polybrene, polyornithine, etc., or the like. The DNA may be single or double stranded DNA, linear or circular. The various techniques for transforming mammalian cells are well known (see Keown et al., Methods Enzymol. (1989), Keown et al., Methods Enzymol. 185:527-537 (1990); Mansour et al., Nature 336:348-352, (1988); all of which are herein incorporated by reference in their entirety).

(h) Insect Constructs and Transformed Insect Cells

The present invention also relates to an insect recombinant expression vectors comprising exogenous genetic material. The present invention also relates to an insect cell comprising an insect recombinant vector. The present invention also relates to methods for obtaining a recombinant insect host cell, comprising introducing into an insect cell exogenous genetic material.

The insect recombinant vector may be any vector which can be conveniently subjected to recombinant DNA procedures and can bring about the expression of the nucleic acid sequence. The choice of a vector will typically depend on the compatibility of the vector with the insect host cell into which the vector is to be introduced. The vector may be a linear or a closed circular plasmid. The vector system may be a single vector or plasmid or two or more vectors or plasmids which together contain the total DNA to be introduced into the genome of the insect host. In addition, the insect vector may be an expression vector. Nucleic acid molecules can be suitable inserted into a replication vector for expression in the insect cell under a suitable promoter for insect cells. Many vectors are available for this purpose, and selection of the appropriate vector will depend mainly on the size of the nucleic acid molecule to be inserted into the vector and the particular host cell to be transformed with the vector. Each vector contains various components depending on its function (amplification of DNA or expression of DNA) and the particular host cell with which it is compatible. The vector components for insect cell transformation generally include, but not limited to, one or more of the following: a signal sequence, and origin of replication, one or more marker genes, and an inducible promoter.

The insect vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the insect cell, is integrated into the genome and replicated together with the chromosome(s) into which it has been integrated. For integration, the vector may rely on the nucleic acid sequence of the vector for stable integration of the vector into the genome by homologous or nonhomologous recombination. Alternatively, the vector may contain additional nucleic acid sequences for directing integration by homologous recombination into the genome of the insect host. The additional nucleic acid sequences enable the vector to be integrated into the host cell genome at a precise location(s) in the chromosome(s). To increase the likelihood of integration at a precise location, there should be preferably two nucleic acid sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the insect host cell, and, furthermore, may be non-encoding or encoding sequences.

Baculovirus expression vectors (BEVs) have become important tools for the expression of foreign genes, both for basic research and for the production of proteins with direct clinical applications in human and veterinary medicine (Doerfler, Curr. Top. Microbiol. Immunol. 131: 51-68 (1968); Luckow and Summers, Bio/Technology 6: 47-55 (1988a); Miller, Annual Review of Microbiol. 42: 177-199 (1988); Summers, Curr. Comm. Molecular Biology, Cold Spring Harbor Press, Cold Spring Harbor, N.Y. (1988); all of which are herein incorporated by reference in their entirety). BEVs are recombinant insect viruses in which the coding sequence for a chosen foreign gene has been inserted behind a baculovirus promoter in place of the viral gene, e.g., polyhedrin (Smith and Summers, U.S. Pat. No. 4,745,051, herein incorporated by reference in its entirety).

The use of baculovirus vectors relies upon the host cells being derived from Lepidopteran insects such as Spodoptera frugiperda or Trichoplusia ni. The preferred Spodoptera frugiperda cell line is the cell line Sf9. The Spodoptera frugiperda Sf9 cell line was obtained from American Type Culture Collection (Manassas, Va.) and is assigned accession number ATCC CRL 1711 (Summers and Smith, A Manual of Methods for Baculovirus Vectors and Insect Cell Culture Procedures, Texas Ag. Exper. Station Bulletin No. 1555 (1988), herein incorporated by reference in its entirety). Other insect cell systems, such as the silkworm B. mori may also be used.

The proteins expressed by the BEVs are, therefore, synthesized, modified and transported in host cells derived from Lepidopteran insects. Most of the genes that have been inserted and produced in the baculovirus expression vector system have been derived from vertebrate species. Other baculovirus genes in addition to the polyhedrin promoter may be employed to advantage in a baculovirus expression system. These include immediate-early (alpha), delayed-early (beta), late (gamma), or very late (delta), according to the phase of the viral infection during which they are expressed. The expression of these genes occurs sequentially, probably as the result of a “cascade” mechanism of transcriptional regulation. (Guarino and Summers, J. Virol. 57:563-571 (1986); Guarino and Summers, J. Virol. 61:2091-2099 (1987); Guarino and Summers, Virol. 162:444-451 (1988); all of which are herein incorporated by reference in their entirety).

Insect recombinant vectors are useful as an intermediates for the infection or transformation of insect cell systems. For example, an insect recombinant vector containing a nucleic acid molecule encoding a baculovirus transcriptional promoter followed downstream by an insect signal DNA sequence is capable of directing the secretion of the desired biologically active protein from the insect cell. The vector may utilize a baculovirus transcriptional promoter region derived from any of the over 500 baculoviruses generally infecting insects, such as for example the Orders Lepidoptera, Diptera, Orthoptera, Coleoptera and Hymenoptera, including for example but not limited to the viral DNAs of Autographa californica MNPV, Bombyx mori NPV, Trichoplusia ni MNPV, Rachiplusia ou MNPV or Galleria mellonella MNPV, wherein said baculovirus transcriptional promoter is a baculovirus immediate-early gene IE1 or IEN promoter; an immediate-early gene in combination with a baculovirus delayed-early gene promoter region selected from the group consisting of 39K and a HindIII-k fragment delayed-early gene; or a baculovirus late gene promoter. The immediate-early or delayed-early promoters can be enhanced with transcriptional enhancer elements. The insect signal DNA sequence may code for a signal peptide of a Lepidopteran adipokinetic hormone precursor or a signal peptide of the Manduca sexta adipokinetic hormone precursor (Summers, U.S. Pat. No. 5,155,037; herein incorporated by reference in its entirety). Other insect signal DNA sequences include a signal peptide of the Orthoptera Schistocerca gregaria locust adipokinetic hormone precurser and the Drosophila melanogaster cuticle genes CP1, CP2, CP3 or CP4 or for an insect signal peptide having substantially a similar chemical composition and function (Summers, U.S. Pat. No. 5,155,037).

Insect cells are distinctly different from animal cells. Insects have a unique life cycle and have distinct cellular properties such as the lack of intracellular plasminogen activators in insect cells which are present in vertebrate cells. Another difference is the high expression levels of protein products ranging from 1 to greater than 500 mg/liter and the ease at which cDNA can be cloned into cells (Frasier, In Vitro Cell. Dev. Biol. 25:225 (1989); Summers and Smith, In: A Manual of Methods for Baculovirus Vectors and Insect Cell Culture Procedures, Texas Ag. Exper. Station Bulletin No. 1555 (1988), both of which are incorporated by reference in their entirety).

Recombinant protein expression in insect cells is achieved by viral infection or stable transformation. For viral infection, the desired gene is cloned into bacuilovirus at the site of the wild-type polyhedron gene (Webb and Summers, Technique 2:173 (1990); Bishop and Posse, Adv. Gene Technol. 1:55 (1990); both of which are incorporated by reference in their entirety). The polyhedron gene is a component of a protein coat in occlusions which encapsulate virus particles. Deletion or insertion in the polyhedron gene results the failure to form occlusion bodies. Occlusion negative viruses are morphologically different from occlusion positive viruses and enable one skilled in the art to identify and purify recombinant viruses.

The vectors of present invention preferably contain one or more selectable markers which permit easy selection of transformed cells. A selectable marker is a gene the product of which provides, for example biocide or viral resistance, resistance to heavy metals, prototrophy to auxotrophs, and the like. Selection may be accomplished by co-transformation, e.g., as described in WO 91/17243, a nucleic acid sequence of the present invention may be operably linked to a suitable promoter sequence. The promoter sequence is a nucleic acid sequence which is recognized by the insect host cell for expression of the nucleic acid sequence. The promoter sequence contains transcription and translation control sequences which mediate the expression of the protein or fragment thereof. The promoter may be any nucleic acid sequence which-shows transcriptional activity in the insect host cell of choice and may be obtained from genes encoding polypeptides either homologous or heterologous to the host cell.

For example, a nucleic acid molecule encoding a C. vulgaris protein homologue or fragment thereof may also be operably linked to a suitable leader sequence. A leader sequence is a non-translated region of a mRNA which is important for translation by the insect host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence which is functional in the insect host cell of choice may be used in the present invention.

A polyadenylation sequence may also be operably linked to the 3′ terminus of the nucleic acid sequence of the present invention. The polyadenylation sequence is a sequence which when transcribed is recognized by the insect host to add polyadenosine residues to transcribed mRNA. The polyadenylation sequence may be native to the nucleic acid sequence encoding the protein or fragment thereof or may be obtained from foreign sources. Any polyadenylation sequence which is functional in the fungal host of choice may be used in the present invention.

To avoid the necessity of disrupting the cell to obtain the protein or fragment thereof, and to minimize the amount of possible degradation of the expressed polypeptide within the cell, it is preferred that expression of the polypeptide gene gives rise to a product secreted outside the cell. To this end, the protein or fragment thereof of the present invention may be linked to a signal peptide linked to the amino terminus of the protein or fragment thereof. A signal peptide is an amino acid sequence which permits the secretion of the protein or fragment thereof from the insect host into the culture medium. The signal peptide may be native to the protein or fragment thereof of the invention or may be obtained from foreign sources. The 5′ end of the coding sequence of the nucleic acid sequence of the present invention may inherently contain a signal peptide coding region naturally linked in translation reading frame with the segment of the coding region which encodes the secreted protein or fragment thereof.

At present, a mode of achieving secretion of a foreign gene product in insect cells is by way of the foreign gene's native signal peptide. Because the foreign genes are usually from non-insect organisms, their signal sequences may be poorly recognized by insect cells, and hence, levels of expression may be suboptimal. However, the efficiency of expression of foreign gene products seems to depend primarily on the characteristics of the foreign protein. On average, nuclear localized or non-structural proteins are most highly expressed, secreted proteins are intermediate, and integral membrane proteins are the least expressed. One factor generally affecting the efficiency of the production of foreign gene products in a heterologous host system is the presence of native signal sequences (also termed presequences, targeting signals, or leader sequences) associated with the foreign gene. The signal sequence is generally coded by a DNA sequence immediately following (5′ to 3) the translation start site of the desired foreign gene.

The expression dependence on the type of signal sequence associated with a gene product can be represented by the following example: If a foreign gene is inserted at a site downstream from the translational start site of the baculovirus polyhedrin gene so as to produce a fusion protein (containing the N-terminus of the polyhedrin structural gene), the fused gene is highly expressed. But less expression is achieved when a foreign gene is inserted in a baculovirus expression vector immediately following the transcriptional start site and totally replacing the polyhedrin structural gene.

Insertions into the region −50 to −1 significantly alter (reduce) steady state transcription which, in turn, reduces translation of the foreign gene product. Use of the pVL941 vector optimizes transcription of foreign genes to the level of the polyhedrin gene transcription. Even though the transcription of a foreign gene may be optimal, optimal translation may vary because of several factors involving processing: signal peptide recognition, mRNA and ribosome binding, glycosylation, disulfide bond formation, sugar processing, oligomerization, for example.

The properties of the insect signal peptide are expected to be more optimal for the efficiency of the translation process in insect cells than those from vertebrate proteins. This phenomenon can generally be explained by the fact that proteins secreted from cells are synthesized as precursor molecules containing hydrophobic N-terminal signal peptides. The signal peptides direct transport of the select protein to its target membrane and are then cleaved by a peptidase on the membrane, such as the endoplasmic reticulum, when the protein passes through it.

Another exemplary insect signal sequence is the sequence encoding for Drosophila cuticle proteins such as CP1, CP2, CP3 or CP4 (Summers, U.S. Pat. No. 5,278,050; herein incorporated by reference in its entirety). Most of the 9 kb region of the Drosophila genome contains genes for the cuticle proteins has been sequenced. Four of the five cuticle genes contain a signal peptide coding sequence interrupted by a short intervening sequence (about 60 base pairs) at a conserved site. Conserved sequences occur in the 5′ mRNA untranslated region, in the adjacent 35 base pairs of upstream flanking sequence and at −200 base pairs from the mRNA start position in each of the cuticle genes.

Standard methods of insect cell culture, cotransfection and preparation of plasmids are set forth in Summers and Smith (Summers and Smith, A Manual of Methods for Baculovirus Vectors and Insect Cell Culture Procedures, Texas Agricultural Experiment Station Bulletin No. 1555, Texas A&M University (1987)). Procedures for the cultivation of viruses and cells are described in Volkman and Summers, J Virol 19: 820-832 (1975) and Volkman et al., J. Virol 19: 820-832 (1976); both of which are herein incorporated by reference in their entirety.

(i) Bacterial Constructs and Transformed Bacterial Cells

The present invention also relates to a bacterial recombinant vector comprising exogenous genetic material. The present invention also relates to a bacteria cell comprising a bacterial recombinant vector. The present invention also relates to methods for obtaining a recombinant bacteria host cell, comprising introducing into a bacterial host cell exogenous genetic material.

The bacterial recombinant vector may be any vector which can be conveniently subjected to recombinant DNA procedures. The choice of a vector will typically depend on the compatibility of the vector with the bacterial host cell into which the vector is to be introduced. The vector may be a linear or a closed circular plasmid. The vector system may be a single vector or plasmid or two or more vectors or plasmids which together contain the total DNA to be introduced into the genome of the bacterial host. In addition, the bacterial vector may be an expression vector. Nucleic acid molecules encoding C. vulgaris protein homologues or fragments thereof can, for example, be suitably inserted into a replicable vector for expression in the bacterium under the control of a suitable promoter for bacteria Many vectors are available for this purpose, and selection of the appropriate vector will depend mainly on the size of the nucleic acid to be inserted into the vector and the particular host cell to be transformed with the vector. Each vector contains various components depending on its function (amplification of DNA or expression of DNA) and the particular host cell with which it is compatible. The vector components for bacterial transformation generally include, but are not limited to, one or more of the following: a signal sequence, an origin of replication, one or more marker genes, and an inducible promoter.

In general, plasmid vectors containing replicon and control sequences that are derived from species compatible with the host cell are used in connection with bacterial hosts. The vector ordinarily carries a replication site, as well as marking sequences that are capable of providing phenotypic selection in transformed cells. For example, E. coli is typically transformed using pBR322, a plasmid derived from an E. coli species (see, e.g., Bolivar et al., Gene 2: 95 (1977); herein incorporated by reference in its entirety). pBR322 contains genes for ampicillin and tetracycline resistance and thus provides easy means for identifying transformed cells. The pBR322 plasmid, or other microbial plasmid or phage, also generally contains, or is modified to contain, promoters that can be used by the microbial organism for expression of the selectable marker genes.

Nucleic acid molecules encoding C. vulgaris protein homologues or fragments thereof may be expressed not only directly, but also as a fusion with another polypeptide, preferably a signal sequence or other polypeptide having a specific cleavage site at the N-terminus of the mature polypeptide. In general, the signal sequence may be a component of the vector, or it may be a part of the polypeptide DNA that is inserted into the vector. The heterologous signal sequence selected should be one that is recognized and processed (i.e., cleaved by a signal peptidase) by the host cell. For bacterial host cells that do not recognize and process the native polypeptide signal sequence, the signal sequence is substituted by a bacterial signal sequence selected, for example, from the group consisting of the alkaline phosphatase, penicillinase, lpp, or heat-stable enterotoxin II leaders.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria The origin of replication from the plasmid pBR322 is suitable for most Gram-negative bacteria.

Expression and cloning vectors also generally contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous gene homologue or fragment thereof produce a protein conferring drug resistance and thus survive the selection regimen.

The expression vector for producing a polypeptide can also contains an inducible promoter that is recognized by the host bacterial organism and is operably linked to the nucleic acid encoding, for example, a C. vulgaris protein homologue or fragment thereof of interest. Inducible promoters suitable for use with bacterial hosts include the beta-lactamase and lactose promoter systems (Chang et al., Nature 275: 615 (1978); Goeddel et al., Nature 281: 544 (1979); both of which are herein incorporated by reference in their entirety), the arabinose promoter system (Guzman et al., J. Bacteriol. 174: 7716-7728 (1992); herein incorporated by reference in its entirety), alkaline phosphatase, a tryptophan (trp) promoter system (Goeddel, Nucleic Acids Res. 8: 4057 (1980); EP 36,776; both of which are herein incorporated by reference in their entirety) and hybrid promoters such as the tac promoter (deBoer et al., Proc. Natl. Acad. Sci. USA 80: 21-25 (1983); herein incorporated by reference in its entirety). However, other known bacterial inducible promoters are suitable (Siebenlist et al., Cell 20:269 (1980); herein incorporated by reference in its entirety).

Promoters for use in bacterial systems also generally contain a Shine-Dalgarno (S.D.) sequence operably linked to the DNA encoding the polypeptide of interest. The promoter can be removed from the bacterial source DNA by restriction enzyme digestion and inserted into the vector containing the desired DNA.

Construction of suitable vectors containing one or more of the above-listed components employs standard ligation techniques. Isolated plasmids or DNA fragments are cleaved, tailored, and re-ligated in the form desired to generate the plasmids required. Examples of available bacterial expression vectors include, but are not limited to, the multifunctional E. coli cloning and expression vectors such as Bluescript Registered™ (Stratagene, La Jolla, Calif.), in which, for example, encoding a C. vulgaris protein homologue or fragment thereof, may be ligated into the vector in frame with sequences for the amino-terminal Met and the subsequent 7 residues of beta-galactosidase so that a hybrid protein is produced; pIN vectors (Van Heeke and Schuster J. Biol. Chem. 264: 5503-5509 (1989). Herein incorporated by reference in its entirety); and the like. pGEX vectors (Promega, Madison Wis.) may also be used to express foreign polypeptides as fusion proteins with glutathione 5-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned polypeptide of interest can be released from the GST moiety at will.

Suitable host bacteria for a bacterial vector include archaebacteria and eubacteria, especially eubacteria, and most preferably Enterobacteriaceae. Examples of useful bacteria include Escherichia, Enterobacter, Azotobacter, Erwinia Bacillus, Pseudomonas, Klebsiella, Proteus, Salmonella, Serratia, Shigella, Rhizobia, Vitreoscilla, and Paracoccus. Suitable E. coli hosts include E. coli W3110 (American Type Culture Collection (ATCC), Manassas, Va.) 27,325), E. coli 294 (ATCC 31,446), E. coli B, and E. coli X1776 (ATCC 31,537). These examples are illustrative rather than limiting. Mutant cells of any of the above-mentioned bacteria may also be employed. It is, of course, necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. E. coli strain W3110 is a preferred host or parent host because it is a common host strain for recombinant DNA product fermentations. Preferably, the host cell should secrete minimal amounts of proteolytic enzymes.

Host cells are transfected and preferably transformed with the above-described vectors and cultured in conventional nutrient media modified as appropriate for inducing promoters, selecting transformants, or amplifying the genes encoding the desired sequences.

Numerous methods of transfection are known to the ordinarily skilled artisan, for example, calcium phosphate and electroporation. Depending on the host cell used, transformation is done using standard techniques appropriate to such cells. The calcium treatment employing calcium chloride, as described in section 1.82 of Sambrook et al., Molecular Cloning: A Laboratory Manual, New York: Cold Spring Harbor Laboratory Press, (1989), is generally used for bacterial cells that contain substantial cell-wall barriers. Another method for transformation employs polyethylene glycol/DMSO, as described in Chung and Miller (Chung and Miller, Nucleic Acids Res. 16: 3580 (1988); herein incorporated by reference in its entirety). Yet another method is the use of the technique termed electroporation.

Bacterial cells used to produce the polypeptide of interest for purposes of this invention are cultured in suitable media in which the promoters for the nucleic acid encoding the heterologous polypeptide can be artificially induced as described generally, e.g., in Sambrook et al., Molecular Cloning: A Laboratory Manual, New York: Cold Spring Harbor Laboratory Press, (1989). Examples of suitable media are given in U.S. Pat. Nos. 5,304,472 and 5,342,763; both of which are incorporated by reference in their entirety.

(j) Computer Media

The nucleotide sequence provided in SEQ ID NO:1, through SEQ ID NO:3519 or fragment thereof, or complement thereof, or a nucleotide sequence at least 90% identical, preferably 95%, identical even more preferably 99% or 100% identical to the sequence provided in SEQ ID NO:1 through SEQ ID NO:3519 or fragment thereof, or complement thereof, can be “provided” in a variety of mediums to facilitate use. Such a medium can also provide a subset thereof in a form that allows a skilled artisan to examine the sequences.

In one application of this embodiment, a nucleotide sequence of the present invention can be recorded on computer readable media As used herein, “computer readable media” refers to any medium that can be read and accessed directly by a computer. Such media include, but are not limited to: magnetic storage media, such as floppy discs, hard disc, storage medium, and magnetic tape: optical storage media such as CD-ROM; electrical storage media such as RAM and ROM; and hybrids of these categories such as magnetic/optical storage media A skilled artisan can readily appreciate how any of the presently known computer readable mediums can be used to create a manufacture comprising computer readable medium having recorded thereon a nucleotide sequence of the present invention.

As used herein, “recorded” refers to a process for storing information on computer readable medium. A skilled artisan can readily adopt any of the presently known methods for recording information on computer readable medium to generate media comprising the nucleotide sequence information of the present invention. A variety of data storage structures are available to a skilled a for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the nucleotide sequence information of the present invention.

By providing one or more of nucleotide sequences of the present invention, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et al., J. Mol. Biol. 215: 403-410 (1990), herein incorporated by reference in its entirety) and BLAZE (Brutlag, et al, Comp. Chem. 17: 203-207 (1993), herein incorporated by reference in its entirety) search algorithms on a Sybase system can be used to identify open reading frames (ORFs) within the genome that contain homology to ORFs or proteins from other organisms. Such ORFs are protein-encoding fragments within the sequences of the present invention and are useful in producing commercially important proteins such as enzymes used in amino acid biosynthesis, metabolism, transcription, translation, RNA processing, nucleic acid and a protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based system are suitable for use in the present invention.

As indicated above, the computer-based systems of the present invention comprise a data storage means having stored therein a nucleotide sequence of the present invention and the necessary hardware means and software means for supporting and implementing a search means. As used herein, “data storage means” refers to memory that can store nucleotide sequence information of the present invention, or a memory access means which can access manufactures having recorded thereon the nucleotide sequence information of the present invention. As used herein, “search means” refers to one or more programs which are implemented on the computer-based system to compare a target sequence or target structural motif with the sequence information stored within the data storage means. Search means are used to identify fragments or regions of the sequence of the present invention that match a particular target sequence or target motif. A variety of known algorithms are disclosed publicly and a variety of commercially available software for conducting search means are available can be used in the computer-based systems of the present invention. Examples of such software include, but are not limited to, MacPattern (EMBL), BLASTIN and BLASTIX (NCBIA). One of the available algorithms or implementing software packages for conducting homology searches can be adapted for use in the present computer-based systems.

The most preferred sequence length of a target sequence is from about 10 to 100 amino acids or from about 30 to 300 nucleotide residues. However, it is well recognized that during searches for commercially important fragments of the nucleic acid molecules of the present invention, such as sequence fragments involved in gene expression and protein processing, may be of shorter length.

As used herein, “a target structural motif,” or “target motif,” refers to any rationally selected sequence or combination of sequences in which the sequences the sequence(s) are chosen based on a three-dimensional configuration which is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences).

Thus, the present invention further provides an input means for receiving a target sequence, a data storage means for storing the target sequences of the present invention sequence identified using a search means as described above, and an output means for outputting the identified homologous sequences. A variety of structural formats for the input and output means can be used to input and output information in the computer-based systems of the present invention. A preferred format for an output means ranks fragments of the sequence of the present invention by varying degrees of homology to the target sequence or target motif. Such presentation provides a skilled artisan with a ranking of sequences which contain various amounts of the target sequence or target motif and identifies the degree of homology contained in the identified fragment.

A variety of comparing means can be used to compare a target sequence or target motif with the data storage means to identify sequence fragments sequence of the present invention. For example, implementing software which implement the BLAST and BLAZE algorithms (Altschul et al., J. Mol. Biol. 215: 40'-410 (1990), herein incorporated by reference in its entirety) can be used to identify open frames within the nucleic acid molecules of the present invention. A skilled artisan can readily recognize that any one of the publicly available homology search programs can be used as the search means for the computer-based systems of the present invention.

### Uses of the Agents of the Present Invention

Nucleic acid molecules and fragments thereof of the present invention may be employed to obtain other nucleic acid molecules from the same species. Such nucleic acid molecules include the nucleic acid molecules that encode the complete coding sequence of a protein and promoters and flanking sequences of such molecules. In addition, such nucleic acid molecules include nucleic acid molecules that encode for other isozymes or gene family members. Such molecules can be readily obtained by using the above-described nucleic acid molecules or fragments thereof to screen cDNA or genomic libraries obtained from C. vulgaris. Methods for forming such libraries are well known in the art.

Nucleic acid molecules and fragments thereof of the present invention may also be employed to obtain other nucleic acid molecules such as nucleic acid homologues. Such homologues include the nucleic acid molecules that encode, in whole or in part, protein homologues of other species, plants or other organisms. Such molecules can be readily obtained by using the above-described nucleic acid molecules or fragments thereof to screen cDNA or genomic libraries. Methods for forming such libraries are well known in the art Such homologue molecules may differ in their nucleotide sequences from those found in one or more of SEQ ID NO:1 through SEQ ID NO:3519 or complements thereof because complete complementarity is not needed for stable hybridization. The nucleic acid molecules of the present invention therefore also include molecules that, although capable of specifically hybridizing with the nucleic acid molecules may lack “complete complementarity” In a particular embodiment, methods or 3′ or 5′ RACE may be used to obtain such sequences (Frohman, M. A. et al., Proc. Natl. Acad. Sci. (USA) 85:8998-9002 (1988); Ohara, O. et al, Proc. Natl. Acad. Sci. (U.S.A.) 86:5673-5677 (1989), both of which are herein incorporated by reference in their entirety).

Any of a variety of methods may be used to obtain one or more of the above-described nucleic acid molecules (Zamechik et al., Proc. Natl. Acad. Sci. (U.S.A.) 83: 4143-4146 (1986); Goodchild et al., Proc. Natl. Acad. Sci. (U.S.A.) 85: 5507-5511 (1988); Wickstrom et al., Proc. Natl. Acad. Sci. (U.S.A.) 85: 1028-1032 (1988); Holt et al., Molec. Cell. Biol. 8: 963-973 (1988); Gerwirtz et al., Science 242: 1303-1306 (1988); Anfossi et al., Proc. Natl. Acad. Sci. (U.S.) 86: 3379-3383 (1989); Becker et al., EMBO J. 8: 3685-3691 (1989); all of which are herein incorporated by reference in their entirety). Automated nucleic acid synthesizers may be employed for this purpose. In lieu of such synthesis, the disclosed nucleic acid molecules may be used to define a pair of primers that can be used with the polymerase chain reaction (Mullis et al., Cold Spring Harbor Symp. Quant. Biol. 51: 263-273 (1986); Erlich et al., European Patent 50,424; European Patent 84,796, European Patent 258,017, European Patent 237,362; Mullis, European Patent 201,184; Mullis et al., U.S. Pat. No. 4,683,202; Erlich, U.S. Pat. No. 4,582,788; and Saiki, R. et al., U.S. Pat. No. 4,683,194, all of which are herein incorporated by reference in their entirety) to amplify and obtain any desired nucleic acid molecule or fragment.

Promoter sequence(s) and other genetic elements including but not limited to transcriptional regulatory elements associated with one or more of the disclosed nucleic acid sequences can also be obtained using the disclosed nucleic acid sequences provided herein. In one embodiment, such sequences are obtained by incubating EST nucleic acid molecules or preferably fragments thereof with members of genomic libraries and recovering clones that hybridize to the EST nucleic acid molecule or fragment thereof. In a second embodiment, methods of “chromosome walking,” or inverse PCR may be used to obtain such sequences (Frohmnan, et al., Proc. Natl. Acad. Sci. (U.S.A.) 85:8998-9002 (1988); Ohara, et al., Proc. Natl. Acad. Si. (U.S.A.) 86: 5673-5677 (1989); Pang et al., Biotechniques, 22(6); 1046-1048 (1977); Huang et al., Methods Mol. Biol. 69: 89-96 (1977); Hartl et al., Methods Mol. Biol. 58: 293-301 (1996), all of which are herein incorporated by reference in their entirety). In one embodiment, the disclosed ESTs are used to identify cDNAs whose analogous genes contain promoters with desirable expression patterns. Isolation and functional analysis of the 5′ flanking promoter sequences of these genes from genomic libraries, for example, using genomic screening methods and PCR techniques would result in the isolation of useful promoters and transcriptional regulatory elements. These methods are known to those of skill in the art and have been described (See for example Birren et al., Genome Analysis:Analyzing DNA, 1, (1997), Cold Spring Harbor Laboratory Press, Cold Spring Harbor, N.Y., herein incorporated by reference in its entirety). Promoters obtained utilizing the ESTs of the present invention could also be modified to affect their control characteristics. Examples of such modifications would include but are not limited to enhancer sequences as reported by Kay et al., Science 236:1299 (1987), herein incorporated by reference in its entirety.

In an aspect of the present invention, one or more of the agents of the present invention may be used to detecting the presence, absence or level of a organism, preferably a green alga and more preferably Chlorella, and even more preferably an C. vulgaris in a sample, In another aspect of the present invention, one or more of the nucleic molecules of the present invention are used to determine the level (i.e., the concentration of mRNA in a sample, etc.) or pattern (i.e., the kinetics of expression, rate of decomposition, stability profile, etc.) of the expression encoded in part or whole by one or more of the nucleic acid molecule of the present invention (collectively, the “Expression Response” of a cell or tissue). As used herein, the Expression Response manifested by a cell or tissue is said to be “altered” if it differs from the Expression Response of cells or tissues of organisms not exhibiting the phenotype. To determine whether a Expression Response is altered, the Expression Response manifested by the cell or tissue of the organism exhibiting the phenotype is compared with that of a similar cell or tissue sample of a organism not exhibiting the phenotype. As will be appreciated, it is not necessary to re-determine the Expression Response of the cell or tissue sample of organisms not exhibiting the phenotype each time such a comparison is made; rather, the Expression Response of a particular organism may be compared with previously obtained values of normal organism. As used herein, the phenotype of the organism is any of one or more characteristics of an organism.

In one sub-aspect, such an analysis is conducted by determining the presence and/or identity of polymorphism(s) by one or more of the nucleic acid molecules of the present invention and more specifically, one or more of the EST nucleic acid molecule or fragment thereof which are associated with phenotype, or a predisposition to phenotype.

Any of a variety of molecules can be used to identify such polymorphism(s). In one embodiment, one or more of the EST nucleic acid molecules (or a sub-fragment thereof) may be employed as a marker nucleic acid molecule to identify such polymorphism(s). Alternatively, such polymorphisms can be detected through the use of a marker nucleic acid molecule or a marker protein that is genetically linked to (i.e., a polynucleotide that co-segregates with) such polymorphism(s).

In an alternative embodiment, such polymorphisms can be detected through the use of a marker nucleic acid molecule that is physically linked to such polymorphism(s). For this purpose, marker nucleic acid molecules comprising a nucleotide sequence of a polynucleotide located within 1 mb of the polymorphism(s), and more preferably within 100 kb of the polymorphism(s), and most preferably within 10 kb of the polymorphism(s) can be employed.

The genomes of animals and plants naturally undergo spontaneous mutation in the course of their continuing evolution (Gusella, Ann. Rev. Biochem. 55:831-854 (1986), herein incorporated by reference in its entirety). A “polymorphism” is a variation or difference in the sequence of the gene or its flanking regions that arises in some of the members of a species. The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence may exist, and the polymorphism is thus said to be di-allelic. In other cases, the species' population may contain multiple alleles, and the polymorphism is termed tri-allelic, etc. A single gene may have multiple different unrelated polymorphisms. For example, it may have a di-allelic polymorphism at one site, and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STR5) that include tandem di- or tri-nucleotide repeated motifs of nucleotides. Polymorphisms characterized by such tandem repeats are referred to as “variable number tandem repeat” (“VNTR”) polymorphisms. VNTRs have been used in identity analysis (Weber, U.S. Pat. No. 5,075,217; Armour, et al., FEBS Left. 307:113-115 (1992); Jones, et al., Eur. J. Haematol. 39:144-147 (1987); Horn, et al., PCT Application WO91/14003; Jeffreys, European Patent Application 370,719; Jeffreys, U.S. Pat. No. 5,175,082; Jeffreys. et al, Amer. J. Hum. Genet. 39:11-24 (1986); Jeffreys. et al., Nature 316:76-79 (1985); Gray, et al., Proc. R Acad Soc. Lond. 243:241-253 (1991); Moore, et al., Genomics 10:654-660 (1991); Jeffreys, et al., Anim. Genet. 18:1-15 (1987); Hillel, et al., Anim. Genet. 20:145-155 (1989); Hillel, et al., Genet. 124:783-789 (1990), all of which are herein incorporated by reference in their entirety).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it Such amplified molecules can be readily detected by gel electrophoresis or other means.

The most preferred method of achieving such amplification employs the polymerase chain reaction (“PCR”) (Mullis, et al., Cold Spring Harbor Symp. Quant. Biol. 51:263-273 (1986); Erlich, et al., European Patent Appln. 50,424; European Patent Appln. 84,796, European Patent Application 258,017, European Patent Appln. 237,362; Mullis, European Patent Appln. 201,184; Mullis, et al., U.S. Pat. No. 4,683,202; Erlich., U.S. Pat. No. 4,582,788; and Saiki, et al., U.S. Pat. No. 4,683,194, all of which are herein incorporated by reference in their entirety), using primer pairs that are capable of hybridizing to the proximal sequences that define a polymorphism in its double-stranded form.

In lieu of PCR, alternative methods, such as the “Ligase Chain Reaction” (“LCR”) may be used (Barany, Proc. Natl. Acad. Sci. (U.S.A.) 88:189-193 (1991), herein incorporated by reference in its entirety). LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide. Alternatively, the oligonucleotides may be selected such that they do not include the polymorphic site (see, Segev, PCT Application WO 90/01069, herein incorporated by reference in its entirety).

The “Oligonucleotide Ligation Assay” (“OLA”) may alternatively be employed (Landegren, et al., Science 241:1077-1080 (1988), herein incorporated by reference in its entirety). The OLA protocol uses two oligonucleotides which are designed to be capable of hybridizing to abutting sequences of a single strand of a target OLA, like LCR, is particularly suited for the detection of point mutations. Unlike LCR, however, OLA results in “linear” rather than exponential amplification of the target sequence.

Nickerson, et al. have described a nucleic acid detection assay that combines attributes of PCR and OLA (Nickerson, et al., Proc. Natl. Acad Sci (U.S.A.) 87:8923-8927 (1990), herein incorporated by reference in its entirety). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Schemes based on ligation of two (or more) oligonucleotides in the presence of nucleic acid having the sequence of the resulting “di-oligonucleotide”, thereby amplifying the di-oligonucleotide, are also known (Wu, et al., Genomics 4:560 (1989), herein incorporated by reference in its entirety), and may be readily adapted to the purposes of the present invention.

Other known nucleic acid amplification procedures, such as allele-specific oligomers, branched DNA technology, transcription-based amplification systems, or isothermal amplification methods may also be used to amplify and analyze such polymorphisms (Malek, et al., U.S. Pat. No. 5,130,238; Davey, et al., European Patent Application 329,822; Schuster et al., U.S. Pat. No. 5,169,766; Miller, et al, PCT Application WO 89/06700; Kwoh, et al., Proc. Natl. Acad. Sci. (U.S.A.) 86:1173-1177 (1989); Gingeras, et al., PCT Application WO 88/10315; Walker, et al., Proc. Natl. Acad. Sci. (USA) 89:392-396 (1992), all of which are herein incorporated by reference in their entirety).

The identification of a polymorphism can be determined in a variety of ways. By correlating the presence or absence of it in an plant with the presence or absence of a phenotype, it is possible to predict the phenotype of that plant If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and plant genetic analyses (Glassberg, UK Patent Application 2135774; Skolnick, et al., Cytogen. Cell Genet. 32:58-67 (1982); Botstein, et al., Ann. J. Hum. Genet. 32:314-331 (1980); Fischer, et al. PCT Application WO90/13668; Uhlen, PCT Application WO90/11369, all of which are herein incorporated by reference in their entirety).

Polymorphisms can also be identified by Single Strand Conformation Polymorphism (SSCP) analysis. The SSCP technique is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5: 874-879 (1989), both of which are herein incorporated by reference in their entirety). Under denaturing conditions a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis. A number of protocols have been described for SSCP including, but not limited to Lee et al., Anal. Biochem. 205: 289-293 (1992); Suzuki et al., Anal. Biochem. 192: 82-84 (1991); Lo et al., Nucleic Acids Research 20: 1005-1009 (1992); Sarkar et al., Genomics 13: 441-443 (1992), all of which are herein incorporated by reference in their entirety). It is understood that one or more of the nucleic acids of the present invention, may be utilized as markers or probes to detect polymorphisms by SSCP analysis.

Polymorphisms may also be found using a DNA fingerprinting technique called amplified fragment length polymorphism (AFLP), which is based on the selective PCR amplification of restriction fragments from a total digest of genomic DNA to profile that DNA (Vos, et al., Nucleic Acids Res. 23:4407-4414 (1995), herein incorporated by reference in its entirety). This method allows for the specific co-amplification of high numbers of restriction fragments, which can be visualized by PCR without knowledge of the nucleic acid sequence.

AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel.

AFLP analysis has been performed on Salix (Beismann, et al, Mol. Ecol. 6:989-993 (1997); Acinetobacter (Janssen, et al., Int. J. Syst. Bacteriol 47:1179-1187 (1997), both of which are herein incorporated by reference in their entirety), Aeromonas popoffi (Guys, et al., Int. J. Syst. Bacteriol. 47:1165-1171 (1997), herein incorporated by reference in its entirety), rice (McCouch, et al., Plant Mol. Biol. 35:89-99 (1997); Nandi, et al., Mol. Gen. Genet. 255:1-8 (1997); Cho, et al., Genome 39:373-378 (1996), all of which are herein incorporated by reference in their entirety), barley (Hordeum vulgare) (Simons, et al., Genomics 44:61-70 (1997); Waugh, et al., Mol. Gen. Genet. 255:311-321 (1997); Qi, et al., Mol. Gen. Genet. 254:330-336 (1997); Becker, et al., Mol. Gen. Genet. 249:65-73 (1995), all of which are herein incorporated by reference in their entirety), potato (Van der Voort, et al., Mol. Gen. Genet. 255:438-447 (1997); Meksem, et al., Mol. Gen. Genet. 249:74-81 (1995), both of which are herein incorporated by reference in their entirety), Phytophthora infestans (Van der Lee, et al., Fungal Genet. Biol. 21:278-291 (1997), herein incorporated by reference in its entirety), Bacillus anthracis (Keim, et al., J. Bacteriol. 179:818-824 (1997), herein incorporated by reference in its entirety), Astragalus cremnophylax (Travis, et al., Mol. Ecol, 5:735-745 (1996), herein incorporated by reference in its entirety), Arabidopsis (Cnops, et al., Mol. Gen. Genet. 253:32-41 (1996), herein incorporated by reference in its entirety), Escherichia coli (Lin, et al., Nucleic Acids Res. 24:3649-3650 (1996), herein incorporated by reference in its entirety), Aeromonas (guys, et al., Int. J. Syst. Bacteriol. 46:572-580 (1996), herein incorporated by reference in its entirety), nematode (Folkertsma, et al., Mol. Plant. Microbe Interact. 9:47-54 (1996), herein incorporated by reference in its entirety), tomato (Thomas, et al., Plant J 8:785-794 (1995), herein incorporated by reference in its entirety), and human (Latorra, et al., PCR Methods Appl. 3:351-358 (1994), herein incorporated by reference in its entirety). AFLP analysis has also been used for fingerprinting mRNA (Money, et al, Nucleic Acids Res. 24:2616-2617 (1996); Bachem, et al, Plant J. 9:745-753 (1996), both of which are herein incorporated by reference in their entirety). It is understood that one or more of the nucleic acid molecules of the present invention, may be utilized as markers or probes to detect polymorphisms by AFLP analysis for fingerprinting mRNA.

Polymorphisms may also be found using random amplified polymorphic DNA (RAPD) (Williams et al., Nucl. Acids Res. 18: 6531-6535 (1990), herein incorporated by reference in its entirety) and cleaveable amplified polymorphic sequences (CAPS) (Lyamichev et al., Science 260: 778-783 (1993), herein incorporated by reference in its entirety). It is understood that one or more of the nucleic acid molecules of the present invention, may be utilized as markers or probes to detect polymorphisms by RAPD or CAPS analysis.

Polymorphisms are useful, through linkage analysis, to define the genetic distances or physical distances between polymorphic traits. A physical map or ordered array of genomic DNA fragments in the desired region containing the gene may be used to characterize and isolate genes corresponding to desirable traits. For this purpose, yeast artificial chromosomes (YACs), bacterial artificial chromosomes (BACs), and cosmids are appropriate vectors for cloning large segments of DNA molecules. Although fewer clones are needed to make a contig for a specific genomic region by using YACs (Agyare et al., Genome Res. 7: 1-9 (1997); James et al., Genomics 32: 425-430 (1996), both of which are herein incorporated by reference in their entirety), chimerism in the inserted DNA fragment can arise. Cosmids are convenient for handling smaller-size DNA molecules and may be used for transformation in developing transgenic plants. BACs also carry DNA fragments and are less prone to chimerism.

Through genetic mapping, a fine scale linkage map can be developed using DNA markers, and, then, a genomic DNA library of large-sized fragments can be screened with molecular markers linked to the desired trait Molecular markers are advantageous for agronomic traits that are otherwise difficult to tag, such as resistance to pathogens, insects and nematodes, tolerance to abiotic stresses, quality parameters and quantitative traits. The essential requirements for marker-assisted selection in a plant breeding program are: (1) the marker(s) should co-segregate or be closely linked with the desired trait; (2) an efficient means of screening large populations for the molecular marker(s) should be available; and (3) the screening technique should have high reproducibility across laboratories, be economical to use and be user-friendly. Molecular marker studies using near-isogenic lines (NILs) (Martin et al., Proc. Natl. Acad. Sci. (U.S.A.). 88: 2336-2340 (1991); Young et al., Genetics 120: 579-585. (1988), both of which are herein incorporated by reference in their entirety), bulked segregant analysis (Michelmore et al., Proc. Natl. Acad. Sci. (U.S.A.) 88: 9828-9832 (1991), herein incorporated by reference in its entirety) or recombinant inbred lines (Mohan et al., Theor. Appl. Genet. 87: 782-788 (1994), herein incorporated by reference in its entirety) have been used to map genes in different plant species (Coe and Neuffer, In: Genetic maps: locus maps of complex genomes, ed. S. J. O'Brien, Cold Spring Harbor Laboratory, Cold Spring Harbor, N.Y., 157-189 (1993), herein incorporated by reference in its entirety). It is understood that one or more of the nucleic acid molecules of the present invention may be used as molecular markers.

In accordance with this aspect of the present invention, a sample nucleic acid is obtained from cells. Any source of nucleic acid may be used. Preferably, the nucleic acid is genomic DNA. The nucleic acid is subjected to restriction endonuclease digestion. For example, one or more EST nucleic acid molecule or fragment thereof can be used as a probe in accordance with the above-described polymorphic methods. The polymorphism obtained in this approach can then be cloned to identify the mutation at the coding region which alters the protein's structure or regulatory region of the gene which affects its expression level.

In one aspect of the present invention, an evaluation can be conducted to determine whether a particular mRNA molecule is present. One or more of the nucleic acid molecules of the present invention, preferably one or more of the EST nucleic acid molecules of the present invention are utilized to detect the presence or quantity of the mRNA species. Such molecules are then incubated with cell or tissue extracts of a plant under conditions sufficient to permit nucleic acid hybridization. The detection of double-stranded probe-mRNA hybrid molecules is indicative of the presence of the mRNA; the amount of such hybrid formed is proportional to the amount of mRNA. Thus, such probes may be used to ascertain the level and extent of the mRNA production in a plant's cells or tissues. Such nucleic acid hybridization may be conducted under quantitative conditions (thereby providing a numerical value of the amount of the mRNA present). Alternatively, the assay may be conducted as a qualitative assay that indicates either that the mRNA is present, or that its level exceeds a user set, predefined value.

A principle of in situ hybridization is that a labeled, single-stranded nucleic acid probe will hybridize to a complementary strand of cellular DNA or RNA and, under the appropriate conditions, these molecules will form a stable hybrid. When nucleic acid hybridization is combined with histological techniques, specific DNA or RNA sequences can be identified within a single cell. An advantage of in situ hybridization over more conventional techniques for the detection of nucleic acids is that it allows an investigator to determine the precise spatial population (Angerer et al., Dev. Biol. 101: 477-484 (1984); Angerer et al., Dev. Biol. 112: 157-166 (1985); Dixon et al., EMBO J. 10: 1317-1324 (1991), all of which are herein incorporated by reference in their entirety). In situ hybridization may be used to measure the steady-state level of RNA accumulation. It is a sensitive technique and RNA sequences present in as few as 5-10 copies per cell can be detected (Hardin et al., J. Mol. Biol. 202: 417-431. (1989), herein incorporated by reference in its entirety). A number of protocols have been devised for in situ hybridization, each with tissue preparation, hybridization, and washing conditions (Meyerowitz, Plant Mol. Biol. Rep. 5: 242-250 (1987); Cox and Goldberg, In: Plant Molecular Biology: A Practical Approach (ed. C. H. Shaw), pp. 1-35. IRL Press, Oxford (1988); Raikhel et al., In situ RNA hybridization in plant tissues. In Plant Molecular Biology Manual, vol. B9: 1-32. Kluwer Academic Publisher, Dordrecht, Belgium (1989), all of which are herein incorporated by reference in their entirety).

In situ hybridization also allows for the localization of proteins within a tissue or cell (Wilkinson, In Situ Hybridization, Oxford University Press, Oxford (1992); Langdale, In Situ Hybridization 165-179 In: The Maize Handbook, eds. Freeling and Walbot, Springer-Verlag, New York (1994), both of which are herein incorporated by reference in their entirety). It is understood that one or more of the molecules of the present invention, preferably one or more of the EST nucleic acid molecules of the present invention or one or more of the antibodies of the present invention may be utilized to detect the expression level or pattern of a protein or mRNA thereof by in situ hybridization.

Fluorescent in situ hybridization also enables the localization of a particular DNA sequence along a chromosome which is useful, among other uses, for gene mapping, following chromosomes in hybrid lines or detecting chromosomes with translocations, transversions or deletions. In situ hybridization has been used to identify chromosomes in several plant species (Griffor et al., Plant Mol. Biol. 17: 101-109 (1991); Gustafson et al., Proc. Nat'l. Acad. Sci. (U.S.A.). 87:1899-1902 (1990); Mukai and Gill, Genome 34: 448-452. (1991); Schwarzacher and Heslop-Harnison, Genome 34: 317-323 (1991); Wang et al., Jpn. J. Genet. 66: 313-316 (1991); Parra and Windle, Nature Genetics, 5: 17-21 (1993), all of which are herein incorporated by reference in their entirety). It is understood that the nucleic acid molecules of the present invention may be used as probes or markers to localize sequences along a chromosome.

It is also understood that one or more of the molecules of the present invention, preferably one or more of the EST nucleic acid molecules of the present invention or one or more of the antibodies of the present invention may be utilized to detect the expression level or pattern of a protein or mRNA thereof by in situ hybridization.

Further, it is also understood that any of the nucleic acid molecules of the present invention may be used as marker nucleic acids and or probes in connection with methods that require probes or marker nucleic acids. As used herein, a probe is an agent that is utilized to determine an attribute or feature (e.g. presence or absence, location, correlation, identity, etc.) or a molecule, cell, tissue or plant. As used herein, a marker nucleic acid is a nucleic acid molecule that is utilized to determine an attribute or feature (e.g., presence or absence, location, correlation, etc.) or a molecule, cell, tissue or plant.

Nucleic acid molecules of the present invention can be used to monitor expression. A microarray-based method for high-throughput monitoring of gene expression may be utilized to measure gene-specific hybridization targets. This ‘chip’-based approach involves using microarrays of nucleic acid molecules as gene-specific hybridization targets to quantitatively measure expression of the corresponding genes (Schena et al., Science 270: 467-470 (1995); Shalon, Ph.D. Thesis, Stanford University (1996), both of which are herein incorporated by reference in their entirety). Every nucleotide in a large sequence can be queried at the same time. Hybridization can be used to efficiently analyze nucleotide sequences.

Several microarray methods have been described. One method compares the sequences to be analyzed by hybridization to a set of oligonucleotides or cDNA molecules representing all possible subsequences (Bains and Smith, J. Theor. Biol. 135: 303 (1989), herein incorporated by reference in its entirety). A second method hybridizes the sample to an array of oligonucleotide or cDNA probes. An array consisting of oligonucleotides or cDNA molecules complementary to subsequences of a target sequence can be used to determine the identity of a target sequence, measure its amount, and detect differences between the target and a reference sequence. Nucleic acid molecules microarrays may also be screened with protein molecules or fragments thereof to determine nucleic acid molecules that specifically bind protein molecules or fragments thereof.

The microarray approach may also be used with polypeptide targets (U.S. Pat. No. 5,445,934; U.S. Pat. No. 5,143,854; U.S. Pat. No. 5,079,600; U.S. Pat. No. 4,923,901, all of which are herein incorporated by reference in their entirety). Essentially, polypeptides are synthesized on a substrate (microarray) and these polypeptides can be screened with either protein molecules or fragments thereof or nucleic acid molecules in order to screen for either protein molecules or fragments thereof or nucleic acid molecules that specifically bind the target polypeptides (Fodor et al., Science 251: 767-773 (1991), herein incorporated by reference in its entirety).

It is understood that one or more of the molecules of the present invention, preferably one or more of the nucleic acid molecules or protein molecules or fragments thereof of the present invention may be utilized in a microarray based method. In a preferred embodiment of the present invention, one or more of the C. vulgaris nucleic acid molecules or protein molecules or fragments thereof of the present invention may be utilized in a microarray based method. A particular preferred microarray embodiment of the present invention is a microarray comprising nucleic acid molecules encoding genes or fragments thereof that are homologues of known genes or nucleic acid molecules that comprise genes or fragment thereof that elicit only limited or no matches to known genes. A further preferred microarray embodiment of the present invention is a microarray comprising nucleic acid molecules having genes or fragments thereof that are homologues of known genes and nucleic acid molecules that comprise genes or fragment thereof that elicit only limited or no matches to known genes.

Nucleic acid molecules of the present invention may be used in site directed mutagenesis. Site directed mutagenesis may be utilized to modify nucleic acid sequences, particularly as it is a technique that allows one or more of the amino acids encoded by a nucleic acid molecule to be altered (e.g. a threonine to be replaced by a methionine). Three basic methods for site-directed mutagenesis are often employed. These are cassette mutagenesis (Wells et al., Gene 34: 315-23 (1985), herein incorporated by reference in its entirety), primer extension (Gilliam et al., Gene 12:129-137 (1980); Zoller and Smith, Methods Enzymol. 100: 468-500 (1983); Dalbadie-McFarland et al., Proc. Natl. Acad. Sci. (U.S.A.). 79: 6409-6413 (1982), all of which are herein incorporated by reference in their entirety) and methods based upon PCR (Scharf et al., Science 233: 1076-1078 (1986); Higuchi et al., Nucleic Acids Res. 16: 7351-7367 (1988), both of which are herein incorporated by reference in their entirety). Site-directed mutagenesis approaches are also described in EP 0 385 962, EP 0 359 472, and PCT Patent Application WO 93/07278, all of which are herein incorporated by reference by reference in their entirety.

Site-directed mutagenesis strategies have been applied to plants for both in vitro as well as in vivo site-directed mutagenesis (Lanz et al., J. Biol. Chem. 266: 9971-9976 (1991); Kovgan and Zhdanov, Biotekhnologjya 5: 148-154, No. 207160n, Chemical Abstracts 110: 225 (1989); Ge et al., Proc. Natl. Acad. Sci. (USA) 86: 4037-4041 (1989); Zhu et al., J. Biol. Chen 271: 18494-18498 (1996); Chu et al., Biochemistry 33: 6150-6157 (1994), Small et al., EMBO J. 11: 1291-1296 (1992); Cho et al., Mol. Biotechnol. 8: 13-16 (1997); Kita et al., J. Biol. Chem. 271: 26529-26535 (1996); Jin et al., Mol. Microbiol. 7: 555-562 (1993); Hatfield and Viersta, J. Biol. Chem. 267:14799-14803 (1992); Zhao et al., Biochemistry 31: 5093-5099 (1992), all of which are herein incorporated by reference in their entirety).

Any of the nucleic acid molecules of the present invention may either be modified by site-directed mutagenesis or used as, for example, nucleic acid molecules that are used to target other nucleic acid molecules for modification. It is understood that mutants with more than one altered nucleotide can be constructed using techniques that practitioners skilled in the art are familiar with such as isolating restriction fragments and ligating such fragments into an expression vector (see, for example, Sambrook et al., Molecular Cloning. A Laboratory Manual, Cold Spring Harbor Press (1989)). In a preferred embodiment of the present invention, one or more of the nucleic acid molecules or fragments thereof of the present invention may be modified by site-directed mutagenesis.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see for example, Sambrook et al., Molecular Cloning. A Laboratory Manual, Cold Spring Harbor Press (1989); Mailga et al., Methods in Plant Molecular Biology, Cold Spring Harbor Press (1995); Birren et al., Genome Analysis Analyzing DNA, 1, Cold Spring Harbor, N.Y., all of which are herein incorporated by reference in their entirety).

Having now generally described the invention, the same will be more readily understood through reference to the following examples which are provided by way of illustration, and are not intended to be limiting of the present invention, unless specified.

### EXAMPLE 1

The cDNA library LIB191 is prepared from the cultures of the eukaryotic green microalgae Chlorella vulgaris C-265. Chlorella cultures were grown in SUN basal salts media supplemented with 15 mM ammonia chloride and 10 mM glucose. The culture is grown with shaking in the presence of light to a high turbidity. Supplemental glucose is added to the media to a concentration of 50 mM and the culture is induced by the additional glucose for 24 hours and harvested. The glucose induction of the cells is confirmed by assaying for the enzyme Sucrose Phosphate Synthase known to be glucose induced. Total RNA is isolated using standard methods and precipitated with LiCl. Poly A+mRNA is purified by oligodT chromatography for use in library construction in pSPORT plasmid.

For the construction of the cDNA library of the present invention, the Superscript™ Plasmid System for cDNA synthesis and Plasmid Cloning (Gibco BRL, Life Technologies, Gaithersburg, Md.) or similar system, following the conditions suggested by the manufacturer, is used. cDNA size fractionation columns from Gibco BRL (Gibco BRL, Life Technologies, Gaithersburg, Md.) are used for size selection of cDNA inserts. Clones are selected and the plasmid DNA is isolated using a commercially available kit.

The quality of the cDNA libraries is determined by examining the cDNA insert size, and also by sequence analysis of a random selection an appropriate number of clones from the library.

### EXAMPLE 2

The cDNA library of the present invention, LIB 191, is plated on LB agar containing the appropriate antibiotics for selection and incubated at 37° C. for a sufficient time to allow the growth of individual colonies. Single colonies are individually placed in each well of 96-well microtiter plates containing LB liquid including the selective antibiotics. The plates are incubated overnight at approximately 37° C. with gentle shaking to promote growth of the cultures. The plasmid DNA is isolated from each clone using a commercially available kit such as Qiaprep plasmid isolation kits, using the conditions recommended by the manufacturer (Qiagen Inc., Santa Clarita, Calif.). A variety of plasmid isolation kits are commercially available.

The template plasmid DNA clones are used for subsequent sequencing. For sequencing the cDNA library LIB191, a commercially available sequencing kit, such as the ABI PRISM dRhodamine Terminator Cycle Sequencing Ready Reaction Kit with AmpliTaq® DNA Polymerase, FS, is used under the conditions recommended by the manufacturer (PE Applied Biosystems, Foster City, Calif.). The ESTs of the present invention are generated by sequencing initiated from the 5′ end of each cDNA clone.

Two basic methods can be used for DNA sequencing, the chain termination method of Sanger et al., Proc. Natl. Acad. Sci. (U.S.A.) 74: 5463-5467 (1977), herein incorporated by reference in its entirety and the chemical degradation method of Maxam and Gilbert, Proc. Natl. Acad. Sci. (U.S.A.) 74: 560-564 (1977), herein incorporated by reference in its entirety. Automation and advances in technology such as the replacement of radioisotopes with fluorescence-based sequencing have reduced the effort required to sequence DNA (Craxton, Method 2: 20-26 (1991); Ju et al., Proc. Natl. Acad. Sci. (U.S.A.) 92: 4347-4351 (1995); Tabor and Richardson, Proc. Natl. Acad. Sci. (U.S.A.) 92: 6339-6343 (1995), all of which are herein incorporated by reference in their entirety). Automated sequencers are available from, for example, Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18: 1415-1419 (1990); Smith, Nature 349: 812-813 (1991); Luckey et al., Methods Enzymol. 218: 154-172 (1993); Lu et al., J. Chromatog. A. 680: 497-501 (1994); Carson et al., Anal. Chem. 65: 3219-3226 (1993); Huang et al., Anal. Chem. 64: 2149-2154 (1992); Kheterpal et al., Electrophoresis 17: 1852-1859 (1996); Quesada and Zhang, Electrophoresis 17:1841-1851 (1996); Baba, Yakugaku Zasshi 117: 265-281 (1997), all of which are herein incorporated by reference in their entirety).

A number of sequencing techniques are known in the art, including fluorescence-based sequencing methodologies. These methods have the detection, automation and instrumentation capability necessary for the analysis of large volumes of sequence data Currently, the 377 DNA Sequencer (Perkin-Elmer Corp., Applied Biosystems Div., Foster City, Calif.) allows the most rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al, Genome Analysis: Analyzing DNA, 1, Cold Spring Harbor, N.Y., herein incorporated by reference in its entirety).

1. A substantially purified nucleic acid molecule comprising the nucleic acid sequence of SEQ ID NO: 9 or the complete complement thereof, wherein said nucleic acid molecule encodes an algal protein or fragment thereof.

2. The substantially purified nucleic acid molecule according to claim 1, wherein said algal protein or fragment thereof is a Chlorella vulgaris protein or fragment thereof.

3. (canceled)

4. A transformed cell having a nucleic acid molecule which comprises:
(A) an exogenous promoter region which functions in said cell to cause the production of a mRNA molecule; which is linked to
(B) a structural nucleic acid molecule, wherein said structural nucleic acid molecule comprises the nucleic acid sequence of SEQ ID NO: 9; which is linked to
(C) a 3′ non-translated sequence that functions in said cell to cause termination of transcription and addition of polyadenylated ribonucleotides to a 3′ end of said mRNA molecule.

5. The transformed cell according to claim 4, wherein said cell is selected from the group consisting of an algal cell, a plant cell, a mammalian cell, a bacterial cell, a fungal cell and an insect cell.

6. The transformed cell according to claim 4, wherein said cell is an algal cell.

7. The transformed cell according to claim 6, wherein said cell is a Chlorella vulgaris cell.

8. A substantially purified nucleic acid molecule comprising a nucleic acid sequence wherein said nucleic acid sequence:
(a) hybridizes under high-stringency conditions to a nucleic acid sequence of SEQ ID NO: 9 or the complete complement thereof, or
(b) shares 90% or greater identity with a nucleic acid sequence of SEQ ID NO: 9 or the complete complement thereof.

9. The substantially purified nucleic acid molecule of claim 8, wherein said nucleic acid molecule encodes a algal protein or fragment thereof.

10. The substantially purified nucleic acid molecule according to claim 9, wherein said algal protein or fragment thereof is a Chlorella vulgaris protein or fragment thereof.

11. A substantially purified nucleic acid molecule comprising a nucleic acid sequence that shares between 100% and 90% sequence identity with a nucleic acid sequence of SEQ ID NO: 9 or the complete complement thereof.

12. The substantially purified nucleic acid molecule of claim 11, wherein said nucleic acid sequence shares between 100% and 95% sequence identity with a nucleic acid sequence of SEQ ID NO: 9 or the complete complement thereof.

13. The substantially purified nucleic acid molecule of claim 12, wherein said nucleic acid sequence shares between 100% and 98% sequence identity with a nucleic acid sequence of SEQ ID NO: 9 or the complete complement thereof.

14. The substantially purified nucleic acid molecule of claim 13, wherein said nucleic acid sequence shares between 100% and 99% sequence identity with a nucleic acid sequence of SEQ ID NO: 9 or the complete complement thereof.

15. The substantially purified nucleic acid molecule of claim 14, wherein said nucleic acid sequence shares 100% sequence identity with a nucleic acid sequence of SEQ ID NO: 9 or the complete complement thereof.

16. A transformed cell having a nucleic acid molecule which comprises:
(a) an exogenous promoter region which functions in a said cell to cause the production of an mRNA molecule; which is linked to;
(b) a structural nucleic acid molecule, wherein said structural nucleic acid molecule comprises a nucleic acid sequence, wherein said nucleic acid sequence
(i) hybridizes under high-stringency conditions to the nucleic acid sequence of SEQ ID NO: 9 or the complete complement thereof; or
(ii) shares 90% or greater identity to the nucleic acid sequence of SEQ ID NO: 9 or the complete complement thereof,
which is linked to
(c) a 3′ non-translated sequence that functions in said cell to cause the termination of transcription and the addition of polyadenylated ribonucleotides to said 3′ end of said mRNA molecule.

17. The transformed cell according to claim 16, wherein said nucleic acid sequence is the complete complement of the nucleic acid sequence of SEQ ID NO: 9.

18. The transformed cell according to claim 16, wherein said cell is selected from the group consisting of an algal cell, a plant cell, a mammalian cell, a bacterial cell, a fungal cell and an insect cell.

19. The transformed cell according to claim 18, wherein said cell is an algal cell.

20. The transformed cell according to claim 19, wherein said cell is a Chlorella vulgaris cell.

21. The transformed cell according to claim 18, wherein said cell is a plant cell.
